(19) United States
(12) Patent Application Publication
(10) Pub. No.: US 2014/0017302 A1
(43) Pub. Date: Jan. 16, 2014

(54) OPTIMISED PREPARATIONS OF HIGHLY ADAPTABLE AGGREGATES

(71) Applicant: Gregor Cevc, Gauting (DE)

(72) Inventor: Gregor Cevc, Gauting (DE)

(21) Appl. No.: 14/006,952

(22) PCT Filed: Mar. 21, 2012
(86) PCT No.: PCT/EP2012/055040

§ 371 Date: Sep. 23, 2013

Related U.S. Application Data
(60) Provisional application No. 61/454,742, filed on Mar. 21, 2011.

(51) Int. Cl. A61K 9/127

(52) U.S. Cl. CPC A61K 9/127
USPC 424/450; 514/570; 514/655

(57) ABSTRACT

The present invention describes improved compositions comprising aggregates having a higher adaptability, or deformability, owing to the inclusion of certain hydrophilic additives, including suitable organic ionic compounds. The disclosed compositions demonstrate superior adaptability and stability over otherwise similar compositions lacking the disclosed additives. The invention furthermore provides methods for manufacturing said aggregate preparations, wherein the resulting preparations are useful for applications such as receiving an aggregate payload with active ingredients, for biological agent delivery, and for a noninvasive targeted treatment of localised body regions at or below the application site of said aggregates.

### 1. FIELD OF THE INVENTION

This invention generally relates to formulations useful for vesicular colloid preparations, especially for the purposes of drug delivery, wherein the disclosed compositions are rendered highly adaptable by inclusion of certain predominantly hydrophilic additives, e.g., organic ions. In particular, the present formulations may be suitable for diagnostic and therapeutic applications. Accompanying guidelines useful for designing and manufacturing said formulations are also provided.

### 2. BACKGROUND OF THE INVENTION

The application of a treatment composition to a mammal in a selective and/or a time-controlled fashion is highly desirable. A properly designed and applied composition, such as a vesicular colloid, can effectively fulfil both of these goals. Known aggregates having the form of a bilayer vesicle have been shown to cross, e.g., semipermeable barriers having pores much narrower than the average aggregate diameter, but this requires sufficient aggregate bilayer deformability and stability as well as a sufficient driving force or pressure (Adv Drug Deliv Rev 56: 675; Honeywell-Nguyen et al., 2006 J Liposome Res 16: 273; Cevc & Vierl, 2010 J Contr Rel 141: 277).

Ultradeformable aggregate compositions have been described. One shared feature required by the oldest known such compositions is at least one edge-active substance at a concentration up to 99 mole-% of the substance concentration, which is necessary for solubilizing the aggregate. A recent related study stated that the solubilising component concentration may be below 0.1 mole-% of the aggregate solubilising-concentration, otherwise the hypothetical aggregate solubilisation is reachable only above 100%. Another feature shared by all such exemplified aggregates is a phospholipid (typically phosphatidylcholine) component. Other researchers have described similar preparations, but required the co-presence of at least one lamellar phase-forming compound and one surfactant with a high affinity for water (typically with a Hydrophilicity-Lipophilicity-Balance (HLB)>12) when the disclosed aggregates were phospholipid free, cholesterol free, and ionic amphipathic molecule free.

Another report described aggregates formed from at least three amphipats that synergistically improved the transport of active substances through a semi-permeable barrier, such as the skin. At least one of the three amphipats was required to be a membrane-forming compound (an MFC, or basic bilayer-forming compound, similar to the “lamellar phase” forming compound of the disclosure mentioned in the previous paragraph). The at least two other amphipats were required to be aggregate forming membrane destabilisers (i.e. MDC1 and MDC2, including at least one nonionic surfactant). A closely related study referred to aggregate compositions for delivering a non-steroidal anti-inflammatory agent (i.e. an NSAID) across intact skin and demonstrated that NSAIDs can, but need not, assume the role of MDC. The disclosure also mentioned various additives (e.g., co-solvents, microbicides, thickening agents, buffering salts). However, the disclosure did not address, or even recognize, any benefit of these agents, if any, on the deformability of the resulting aggregates.

A more specialised report described the use of phospholipid-surfactant vesicle compositions for delivering terbinafine, or a pharmaceutically acceptable salt thereof, to the fingernail of a subject in an effective amount for treating onychomycosis. The compositions required a phospholipid and a surfactant, wherein the formulation comprised 0.5-10% terbinafine or a pharmaceutically acceptable salt thereof by weight, 2-10% phospholipid by weight, and 1-5% surfactant by weight, and wherein the molar ratio of phospholipid to surfactant in the formulation was specified to be 1/1 to 5/1. Chloride, bromide, iodide, acetate, and fumarate were singled-out as suitable terbinafine salt forms, without explaining how any these salts might impact terbinafine delivery by or function of the vesicle. Other mentioned pharmaceutically acceptable acids and bases follow closely the “Handbook of Pharmaceutical Salts. Properties, and Use” (Stahl and Wermuth, eds., Wiley-VCH, Zurich, 2002). The study also referred to the inclusion of acetate, lactate, phosphate, and propionate buffers in the described vesicles, but again did not recognize any beneficial difference between them.

Other public disclosures relate to alcohol-enhanced liposomes for improved transdermal drug delivery of a compound to a target location. The liposomal compositions have been stated to comprise 0.5-10 wt.-% phospholipid, at least 20 wt.-% water, 20-50 wt.-% C2, C3 or C4 alcohol (being a mixture of 15-30% ethanol and 5-35% C3 or C4 alcohol), up to 20 wt.-% glycerol, and at least one active ingredient.

Collectively, there are few meaningful choices available to a skilled practitioner aiming to make and/or use adaptable vesicle compositions for effective and noninvasive drug delivery through a physiological barrier. Known aggregates require either a combination of (phospho)lipids and one or more (solubilising and typically nonionic) detergents or alternatively a combination of such lipid(s) and a substantial quantity of (solubilising) lower-alkyl alcohol(s).

What is needed are improved compositions and related methods of use for noninvasive drug delivery with minimal side effects to a mammal and easy to design and use. Such composition should ideally involve hydrophilic additives, such as dissociated salts, to modulate advantageously amphipathic aggregates in a suspension in a manner that optimizes their ability to traverse a barrier such as the skin, and/or provides enhanced stability and utility in a wide variety of diagnostic and/or therapeutic applications and/or affords manufacturing or other commercial advantages.

### 3. SUMMARY OF THE INVENTION

The present invention discloses a variety of aggregate compositions, preferably in a vesicular formulation, with improved “useful properties”, including but not limited to adaptability, an aspect relates to the ability of the aggregate compositions to cross small, micro- or nano-barriers and/or aggregate stability and/or aggregate payload and/or aggregate manufacture. Improved, or an improvement in the aggregate properties refers to a favorable, i.e. desirable, change of any aggregate property usually by a sizeable magnitude, preferably at least by 20%. Some or all of these goals are achieved by altering molecular distribution, such as packing of lipophilic molecules in the aggregate-forming bilayer. As explained herein, the unexpected effectiveness of these compositions is achievable by including at least one kind of hydrophilic additive to amphipat combinations that are often unique and quite distinct from the known aggregate formulations (which were based predominantly, if not exclusively, on phospholipid-surfactant combinations). The selection and quantity of the optimal additive to be used in a composition according to the invention is such that it facilitates a timely additive (re)distribution in aggregates suspension, mainly perpendicular to the bilayer surface. This transverse redistribution of essentially hydrophilic molecules near the bilayer is distinct from the lateral redistribution of lipids used to the same end in the art, and makes the aggregate more adaptable, e.g., due to influences exerted by charge-charge interactions.

An additional aim of the invention concerns the identification of compositions that produce aggregates in the form of deformable bilayer vesicles that are sufficiently adaptable and can readily interact with a porous barrier, such as the skin, to mediate material transfer into underlying peripheral tissue. To meet this goal, the invention additionally provides several different selection tools that are useful for establishing an optimized vesicle formulation.

The first selection tool is mostly qualitative in nature and relies on structural information about a candidate additive used to maximize the bilayer deformability of the vesicular formulation. Potentially suitable additives, such as anionic, cationic and uncharged (e.g. zwitterionic) molecules, are described. The second selection tool is mostly quantitative and defines various and preferred ranges for the partition ratio, dissociation constant, and target concentration(s) of the additives suitable for making and using the preparations of the invention.

An additional aim of the invention is directed to determining an optimal additive(s) for incorporation into the aggregate formulations, in addition to suitable aggregate-forming amphipats or amphipat combinations that occupy an average area per chain (Ac) in the final bilayer of between about 0.40 nm² and about 0.50 nm², or maximally 0.55 nm², and more preferably between 0.42 nm² and 0.48 nm² for the amphipats with about 18 carbon atoms (=C-atoms) per hydrophobic chain, wherein such Ac is influenced by the chosen additive type and concentration thereof.

The aforementioned selection tools can be readily applied to existing (e.g. phospholipid-containing) or to new (e.g. phospholipid-free) deformable and adaptable aggregate (e.g. vesicle) preparations.

Numerous embodiments of the invention authenticate the disclosed selection tools and specify various aggregate compositions comprised of one heterogeneous amphipat product or several different amphipats; aggregates of amphipats with relatively short or long fatty-chains; aggregates with or without cationic or anionic drug cargo; and suspensions in buffers having a wide range of pH values. The invention moreover provides illustrative aggregate preparations meeting at least one the aforementioned goals for one or more non-limiting drug classes including ionisable (anionic) NSAIDs or (cationic) antifungal agents. The invention also describes suitable (counter)ions and pH selection guidelines for use in these compositions.

The invention also identifies suitable manufacturing processes, testing schemes, and suggested schedules for applying the described formulations according to the invention. The disclosed aggregate compositions are suitable for a wide variety of therapeutic and diagnostic purposes such as, but not limited to, use as pharmaceutical drug carriers. The latter can be administered, e.g., on the surface of, or internally applied to, a mammalian body, such as a human body. By modulating bilayer deformability and aggregate adaptability with the selected formulation components, including buffers, microbicides, antioxidants, and the like, elegantly minimalistic and cost optimised preparations of the invention are obtainable.

In certain situations, it may be desirable to create a preparation of the invention with higher carrier-associated drug concentration, or to produce more stable aggregates for a contemplated purpose. To meet these goals, the invention provides guidance as to what type of additive(s) can effectively accelerate the dispersion of a particular amphipat, or combinations thereof, into small suspended aggregates, thus shortening the suspension time by at least 50% compared with the suspension time of a comparable amphipathic mixture in commonly used inorganic buffers.

Monitoring the relative speed of small aggregates formation under constant external stress as a function of certain additive (concentration) can therefore attain the objectives of the invention according to the following tests. First, one selects a desirable amphipat(s) prone to form adjustable aggregates according to the invention. Second, one selects the desired type, ionisation state (if applicable), and concentration of the primary water-soluble components to fine-tune the resulting preparation (allowing for any influences exerted by the other formulation components). Third, as required, one adjusts the relative concentration(s) of all functionally important formulation components according to the invention until the desired aggregate characteristics are reached.

The details of one or more embodiments of the invention are set forth in the description below. Other features, objects, and advantages of the invention will be apparent from the description and in the appended claims.

### 4. BRIEF DESCRIPTION OF THE DRAWINGS

FIG. 1 is a schematic representation of experiments carried out to show an effect of buffer (ionic additive) selection on the vesicularisation time of reference (i.e. containing >90% pure soybean phosphatidylcholine) vesicles and of vesicles loaded with terbinafine as a function of the bulk pH. The results in the main two panels are derived from Table 5. The results of an additional test series, in which an increasing amount of terbinafine was incorporated into phosphatidylcholine vesicles (at 10 wt-%-terbinafine concentration+5 wt.-% ethanol) dispersed in azeleate buffer (0.05 M) are shown in the Inset for pH=4.55±0.1 (diamonds) and pH=3.6±0.1 (down-arrow). The schematic shows at least 50% higher payload (concentration at minimum vesicularisation time: ˜15% at pH=4.55 and ˜20% at pH=3.6) in azeleate buffer as compared to aggregate suspensions in a phosphate-buffer of comparable pH (where such minimum/payload limit is at <10 wt.-%).

### 5. DETAILED DESCRIPTION OF THE INVENTION

Unless defined otherwise, all technical and scientific terms used herein have their plain, general meaning understood to one of ordinary skill in the art in the relevant technical field, including particularly to the terms “aliphatic” (chain), “alkanoyl”, “alkenoyl”, “amphipat” or “amphiphile” or “amphipathic”, “branched”, “halo” or “halide”, “heteroaryl”, “heterocyclic or “heterocyclyl”, “homogeneous”, “lamellar phase”, “pharmacologically acceptable”, “substituent” and to “substitute”.

The term “about”, or “around” when used with a numerical value, means a range surrounding the corresponding numerical value, including the typical measuring error associated with a particular experiment. Unless specifically stated to be, e.g., ±1%, ±2%, ±3%, ±4%, ±5%, ±7.5%, ±10%, ±15%, ±20%, ±25%, ±30%, ±35%, ±40% or any other percentage of the numerical value, the term “about” or “around” used in connection with a particular numerical value generally means ±25%. For imprecisely known or not uniquely defined quantities, this term implies a range of ±50%.

The term “acyl” means a linear hydrocarbon radical with 2 to n (C₂-Cₙ), carbon atoms and comprising a carbonyl group, wherein “n” is typically selected to be a whole integer between 2 and 30.

The term aggregate “adaptability” is herein closely related to “deformability” and can be measured using previously described methods (e.g. Wachter et al., 2008, J. Drug Targeting 16: 611). In principle, most of these methods assess the penetration of a nanoporous, semipermeable barrier by the tested aggregates in a suspension, presuming no significant aggregate fragmentation during penetration. In an alternative method, the kinetics of aggregate fragmentation under greater external stress is studied, e.g., during ultrasonication. An aggregate is considered to have an ultradeformable bilayer for purposes of the invention if its adaptability is close to or at about the highest value achievable without an appreciable, and normally spontaneous, aggregate fragmentation into smaller structures, e.g. micelles. An alternative criterion is reaching at least 5 times, more preferably 10-times, or even more preferably, 20-times shorter enforced vesicularisation time compared with conventional, poorly deformable lipid bilayer vesicles (e.g. the reference fluid-phase liposomes made of >95% pure phosphatidylcholine) under comparable conditions. Confirmation of functional similarity between, or adaptability of, any newly tested formulation and a formulation previously shown to be ultradeformable can prove the point as well.

The term “additive of the invention” means herein a compound that is typically but not necessarily an organic molecule that is not a surfactant, i.e. does not form spherical, rod- or thread-like micelles or bilayers, and which increases significantly, i.e. for the purpose by at least 20%, the adaptability of bilayer aggregates comprising one or more type of amphipats and/or accelerates vesicle aggregate size diminution from the originally at least 2 times larger aggregates under external stress. Particularly suitable additives of the invention are the preferred ions as defined herein and/or other amphipathic substances with log P=−1±3, preferably with log P=1±2.5, and even more preferably with log P=−1±2.

The term “aggregate” as used herein in connection with the term “composition” is also interchangeable with, and refers to an aggregate “preparation” or “formulation”, unless specified otherwise.

The term “alkenyl” means a linear or branched monovalent hydrocarbon radical containing one or several carbon-carbon double bonds in either (the more preferred) “cis” or (the less preferred) “trans” configuration including but not limited to allyl, butenyl, ethenyl, 4-methylbutenyl, propen-1-yl, and propen-2-yl.

The term “alkyl” refers to a linear or branched saturated monovalent hydrocarbon radical that can include one or several substituents and is typically a linear saturated monovalent hydrocarbon radical with 1 to n (general notation: C₁=C1 to Cₙ=Cn), wherein “n” is typically selected to be a whole integer between 1 and 30.

The term “anion” means herein any negatively charged atom or group of atoms, typically soluble in water and having a tendency to migrate to an anode in an electrolytic cell, including combinations and/or substituted forms thereof. Examples include hydroxide and various carbonate ions, dissolved salts of halo-acids, such as halides, hipohalites (e.g. hypochlorite, hypobromite or hypoiodite), halites (e.g. chlorite), halidates (such as chlorate, bromate or iodate), perhalidates (such as perchlorate or periodate), other inorganic at least partially dissociated acids, especially various phosphates (including phosphate proper, phosphonate, phosphinite, phosphonite, phosphite, phosphinate), sulphates (including peroxomonosulphate, sulphate, sulphite, peroxodisulphate, pyrosulphate, dithionate, metabisulphite, dithionite, thiosulphate, tetrathionate), but also permanganate, etc. Cyanate and thiocyanide are more preferred anions for purposes of this invention than the more toxic cyanide.

Particularly useful anions for the purposes of the invention are the conjugate bases of organic poly- or monoprotic Lewis acids, which may be only partially ionised. The latter kind of acid can be an oxoacid and give rise to various monovalent carboxylate ions. The latter kind may be derivatised with at least one and potentially several acyl, alkenyl, alkyl, alkynyl, aralkyl, aryl, cycloalkyl, heterocyclyl, or heteroaryl residues. Exemplary aliphatic carboxylates are short, straight or branched chain carboxylates (preferred are the lower alkyl carboxylates, such as formate, acetate and, when the odor is tolerable, propionate, butyrate and the less odiferous isobutyrate, as well as pentanoate (valerate) and especially methylbutyrate or isovalerate methylpentanoate or isocaproate, diethylacetate or ethylbutanoate, ethylvalerate, 3-ethylpentanoate), methylhexanoate and halido butyrate, pentanoate or hexanoate; moreover, hydroxyacetates, such as citramalate, ribonate or the corresponding charged gamma-lactone derivative(s)), gulonate and the corresponding gulonolactone derivatives, 2,3,4-trihydroxybutanoate, 2,3,4,5-tetrahydroxypentanoate, 2,3,4,5,6-pentahydroxyhexanoate, 2,3,4,5,6,7-hexahydroxyheptanoate, aleurate, 2-hydroxy-3-methylbutyrate, 3-hydroxy-3-methyl butyrate, leucate (i.e., leucic acid salts), 3-hydroxy-3-methylpentanoate, 3- or 4-hydroxybutanoate (i.e. 3-, or 4-hydroxybutyrate), 2.hydroxypentanoate, 3-, 4- or 5-hydroxypentanoate, 2-, 3-, 4-, 5- or 6-hydroxyhexanoate, -heptanoate or -octanoate, etc. up to 3-, 4-, 5-, 6-, 7-, 8- or 9-hydroxydecanoate, the corresponding dihydroxy fatty acid derivatives, 4-hydroxy-2,2-diphenylbutanoate or glycolate (hydroxyacetate), lactate, beta-lactate or lactobionate, 3- or 4-hydroxybutyrate (i.e. beta- or gamma-hydroxybutyrate); dicarbonates, such as tartronate, malate, hydroglutarate, hydroxyadipate, tartrate, 2,3-dihydroxypentanedioate, 2,4-dihydroxypentanedioate, alpha-ketoglutarate, 2,5-dihydroxyhexanedioate, arabinarate galactarate, glucarate (the latter two both corresponding to 2,3,4,5-tetrahydroxyhexanedioate), iduronate, glucuronate, gluconate, glucoheptonate; oxoacetate, pyruvate, 2-oxobutyrate, acetoacetate, levulinate, oxalate, malonate, succinate, glutarate, and oxoglutarate, adipate, pimelate, suberate, azelate (nonanedioate), fumarate, maleate, the corresponding alkyl esters, such as methylmalonate (iso-succinate), ethylmalonate, propylmalonate and butylmalonate, methylsuccinate, or the corresponding ethyl-, propyl- and butyl-derivatives, methyl-, ethyl-, propyl- or butyl-glutarate, methyl-, ethyl-, propyl- or butyladipate, methyl-, ethyl-, and propylpimelate, methyl- and ethylsuberate, methylazelate, the corresponding β-alkyl-isomers, such as methylglutarate, β-methyladipate, β-methylpimelate being relevant as well; furthermore di-branched compounds such as dimethylmalonate, diethyl-, dipropyl- and dibutylmalonate, dimethyl, diethyl- and dipropylsuccinates, dimethyl-, diethyl- and dipropylglutarates (including e.g. 2,2-, 3,3-, or 2,4-dipropylpentanedioate), dimethyl- and diethylhexanedioates, dimethylheptanedioates, vinylogous carboxylate (such as ascorbate or salt of Meldrum's acid), citrate, etc. Any aliphatic chain attached to a carboxylate may be replaced by an aromatic residue, e.g. of aryl, heteroaryl, or heterocyclyl type, as is exemplified by hydroxyacetates tropate (i.e. 2-phenylhydracrylate or 3-hydroxy-2-phenylpropanoate) or benzilate, diflunisil, or by carboxylates such as hippurate, 1-hydroxy-1-cyclopropane carboxylate, sulfurol acetate or 2-(4,5-dihydro-1,3-thiazol-2-ylsulfanyl)acetate, by 2- or 3-thiophenic (=thenoic) acid, 2- or 4-hydroxymandelate, 3-alkoxy-4-hydroxyphenyl)(hydroxy)acetate, e.g. in 3-ethoxy-4-hydroxyphenyl)-(hydroxy)acetate, 3-chloro-4-hydroxymandelate and 4-chloromandelate, 3-hydroxy-4-methoxymandelate, 4-hydroxy-3-methoxymandelate, 3-(2hydroxyphenyl)lactate, 3-(4-hydroxyphenyl)lactate, hexahydroxymandelate, etc. Other suitable carboxylates having at least one cyclic group include 5,6,7,8-tetrahydro-1-naphthoate, 1-hydroxy-2-naphthoate, 1,2,3,4-tetrahydronaphthalene-1,5-(di)carboxylate, camphorate, camphorsulphonate (esp. camphor-10-sulphonate), nicotinate (pyridine-3-carboxylate), the mono- or divalent pamoate (embonate), etc. Further useful aromatic anions of oxoacid type include pyromucate, 3-alkenyl-2-furoate, such as 3-methyl-2-furoate, 3-ethenylfuran-2-carboxylate, 2-(furan-2-ylmethoxy)acetate, methyl-2-(furan-2-ylmethoxy)acetate, 2-(furan-2-ylmethoxy)-3-methylbutanoate, furan-2-ylmethyl formate, 3-propan-2-ylfuran-2-carboxylate, furan-2-carboperoxoate, 2-(furan-2-carbonyloxy)pentanoate, 2- or 3-(furan-2-ylmethoxy)acetate, 2- or 3-(furan-2-ylmethoxy)propanoate, 2- or 3-(furan-2-ylmethoxy)butanoate, furan-2-ylmethyl hydrogen carbonate, 4-(furan-2-ylmethoxy)-4-oxobutanoate, 5-(furan-2-ylmethoxy)-5-oxobutanoate, 4-(furan-2-ylmethoxy)-4-oxopentanoate, 5-(furan-2-ylmethoxy)-5-oxopentanoate, 3- or 5-halidofuran-2-carboxylate (such as 3- or 5-bromo- or 3- or 5-fluorofuran-2-carboxylate), 2,2-difluoro-2-(furan-2-ylmethoxy)acetate, 4-(trifluoromethyl)furan-2-carboxylate, 1-(furan-2-yl)-4-methoxy-3,4-dioxobut-1-en-1-olate, (2-carboxyfuran-3-yl)-methylsilicon salt, hydroxylmethylfuroate and dihydroxymethylfuroate; phenylglycolate, halido- and dihalido-mandelate, p-alkyl-mandelate, p-fluoro-mandelate, trifluoromethylmandelate, O-methoxy-mandelate, O-methyl-mandelate, methyl 2-hydroxy-2-phenylpropanoate, 3,4-dimethoxy-mandelate, 3,4-dihydroxymandelate, phenyllactate, atrolactate; benzoate, 2-, 3-, or 4-hydroxybenzoate, lower alkyl-4-hydroxybenzoate (such as methyl-hydroxybenzoate), lower alkyl-2-hydroxybenzoate (such as methyl salicylate), short-chain 4-alkenoxy-2-hydroxybenzoate, 2-, 3-, 4-, 5- or 6-halido-benzoate, 2-, 3-, 4-, 5-, or 6-halido-hydroxybenzoate, dihalidohydroxybenzoate, trihalidohydroxy-benzoate, ethoxyhydroxybenzoate, hydroxy-alkylbenzoate (such as 5-methyl-salicylate), 3-, 4- or 5-aminosalicylate, 3-hydroxy-4,5-dimethoxybenzoate, vanillate), gamma-resorcylate or gentisate, galliate, 4-acetamidobenzoate, 2-acetamido-benzoate, etc.; benzyl- or phenyl-dioic acids, such as benzylmalonate, benzyl-succinate, benzylfumarate, benzylmaleate or phenylmalonate; butilfenin, indolacetate, and other indole-derivatives with a desirable partition ratio, such as x-hydroxy-cinnamate and dihydroxycinnamate, x-alkenoxy- and dialkenoxy-, x-alkyl- (such as x-methyl) and dimethylcinnamate, coumarate, halidocoumarate, halidocinnamate, dihalidocinnamate, 3-x-halidophenyl)prop-2-enoate, always with x=2, 3, 4, 5, or 6; etc; pyrrolidine-2,4-(di)-carboxylate, pyrrolidine-dithiocarbamate, 1-pyrrolidino-1-cyclohexene, prolinate, halidoprolinate, 1-alkylpyrrolidin-2-carboxylate, 2- or 3-alkylpyrrolidin-2-carboxylate, pidolate, 5-oxopyrrolidine-2-carboxamide, N-alkyl-, such as N-ethyl- or N-propyl-, -5-oxopyrrolidine-2-carboxamide, any other anion compatible with the chosen embodiment and the desired purpose of formulation administration.

Additional conjugate bases suitable for the invention include aliphatic or aromatic phosphate derivatives that can carry one or several acyl, alkenyl, alkyl, alkynyl, aralkyl, aryl, cycloalkyl, heterocyclyl, or heteroaryl groups, and optionally may contain one or several (directly attached (hydroxy) or indirectly attached (alkoxy)) oxygen atoms, and/or nitrogen, sulphur, or halide atoms at any heteroatom or carbon atom that provides a stable aggregate composition, including but not limited to lower alkyl phosphates (such as methyl-, ethyl-, propyl-, butyl-, pentyl-, or hexyl-phosphate and their iso-forms) and the corresponding (mixed) lower dialkyl phosphates, such as dimethylphosphate, diethylphosphate, methylethylphosphate, dipropylphosphate, diisopropylphosphate, etc.; the corresponding substituted mono and dialkylated biphosphates (pyrophosphates), such as isopentenyl pyrophosphate, isopentyl pyrophosphate, diethyl- or dipropylpyrophosphate; the alkyl, aryl or heteroaryl substituted or any stable halido-, hydroxy- or alkoxy-derivative of any such substance, such as 2-hydroxyethanephosphonate, and the branched phosphonates like N-(phosphonomethyl)iminodiacetate or phosphono-hetero-acids, such as 2-carboxyethyl phosphonate (CEPA) or 2-hydroxyphosphonocarboxylate (HPAA).

Also useful according to the invention are the conjugate bases formed from the non-aromatic cyclopentyl- or cyclohexyl-phosphate or -phosphonate, the aromatic benzenephosphate or -phosphonate, the corresponding naphthalene-, p-toluene-, xylene-phospho-conjugates, etc.; furthermore, the aromatic anionic phospho-substitutes such as 2-, 3-, 5-, 6- and especially 4-methoxybenzenephosphonates, 2-, 3-, 5-, 6- and especially 4-alkyl-benzenephosphonates, such as 4-methyl-, 4-ethyl-, 4-propyl-, 4-butyl- or 4-pentyl-benzenephosphonates, the branched chain 4-(2,2-dimethylpropyl)benzenephosphonate, 4-(2-methylpropyl)benzenesulphonate, 4-(3-methylbutyl)benzenephosphonate, related compounds, such as 1,2,3,4-tetrahydronaphthalene-phosphonate or -diphosphonate; 4-ethenylbenzenephosphonate, or 2-aminoethylphosphonate (AEPN), dimethyl methylphosphonate (DMMP), 1-hydroxy ethylidene-1,1-diphosphonate (HEDP), ethylenediamine tetra(methylene phosphonate (EDTMP), tetramethylenediamine tetra(methylene phosphonate) (TDTMP), hexamethylenediamine tetra(methylene phosphonate) (HDTMP), diethylenetriamine penta-(methylene phosphonate) (DTPMP), by way of example; less preferred phosphinates (hypophosphates) and phosphothioates can be substituted in like fashion.

Additional suitable anion components moreover include sulphates, which can be substituted similarly to the phosphates described above, e.g., alkyl sulphonates (alkyl sulphites), mesylate, esylate, propanesulphonate, butanesulphonate or pentanesulphonate or -disulphonate (e.g. ethanedisulphonate=edisylate), the corresponding sulphinates; furthermore all their chemically stable (pluri)oxo-, (pluri)hydroxy- or (pluri)alkoxy-derivatives, also in their halidated forms, such as triflate and the corresponding thiosulphonates; cyclo-sulphonates and -sulphinates, cyclamate, besylate and tosylate, xylenesulphonate, various naphthalenesulphonates (such as napsylate) and -trisulphonates, their (pluri)oxo-, (pluri)hydroxy- or (pluri)alkoxy-substitutes, polystyrene sulphonate (sulphonated polystyrenate) and all corresponding sulphinates. Moreover, the hydrophobic chain can be substituted further, e.g. with a 2-alkenoxyethyl sulphate.

Additional illustrative but not limiting anion examples include alkyl aryl sulphonates, aryl sulphonates, heteroaryl or heterocyclyl sulphonates, and the chemically stable and practically acceptable sulphinates. The correspondingly substituted nitrates or nitrites, and less preferred borates, borites or tetraborates, chromates and selenates, are also suitable for the preparations of the invention. Furthermore, various benzenesulphinates, 4-methylbenzenesulphonoperoxoates, 2-, 3- or 5-halido-4-methylbenzenesulphonates, and (5-methyl-2-sulphophenyl)siliconate; educts and combinations thereof, 2-morpholinoalkanesulphonates, and saccharine=1,1-dioxo-1,2-benzothiazol-3-one, which offer useful proximity of N- and S-atoms, are also suitable anions according to the invention.

Consequently, the term “anionic group” herein includes, inter alia, the charged residue of any of the acid classes referred to herein, such as arsenate, borate, carboxylate, cyanate, phosphate, phosphonate, phosphinate, selenate, sulphate, sulphonate, sulphinate, thiocarboxylate, thyocyanate, thioglycolate, thiosulphate and thiophosphate groups, or homo-combinations (such as biphosphate, biphosphonate, bichromate, bisulphate, bisulphite, mono- or dicarboxylate, or heterocombinations (such as phosphosulphonate, sulphosuccinate, etc.) or substituted forms and combinations thereof.

The term “antifungal” or “antimycotic” (agent) includes, but is not limited to allylamines, such as butenafine, naftifine, or terbinafine and their analogues, candicin, imidazoles, such as bifonazole, butoconazole, clotrimazole, econazole, fenticonazole, isoconazole, ketoconazole, or miconazole plus omoconazole, oxiconazole, sertaconazole, sulconazole, tioconazole), triazoles, such as fluconazole, isavuconazole, or itraconazole plus posaconazole, ravuconazole, voriconazole, terconazole), or amorolfine or griseofulvin, abafungin, amphotericin B, filipin, hamycin, liranaftate, natamycin, nystatin, rimocidin, or tolnaftate. A suitable listing is disclosed in U.S. Ser. No. 12/508,537.

The term “antimicrobial” agent, or microbicide, means at least one, and more frequently a combination of, substance(s) that reduce pathogen count and/or prevent pathogen growth in the preparations if included; pathogens in this context are mainly bacteria, yeast, fungi and mold, plus potentially viruses. Additional potentially useful antimicrobial compounds are listed in “Directory of Microbicides for the Protection of Materials. A Handbook (in two parts, W. Paulus, ed.), Springer, Berlin, 2005.

The term “antioxidant” refers to any substance suppressing oxidation in the described formulations, including but not limited to aromatic amines, ascorbic, kojic and malic acid and their salts, thioglycerol, nordihydroguaiaretic acid (NDGA), p-alkylthio-o-anisidine, a phenol or a phenolic acid; tetrahydroindenoindol; thymol; tocopherol and its derivatives; trolox and the corresponding amide and thiocarboxamide analogues; quinic acid, and vanillin. Also useful are preferentially oxidizable compounds, such as sodium bisulphite, sodium metabisulphite, thiourea, as well as chelating agents, such as EDTA, EGTA, ethyleneglycol-bis-N,N′-tetraacetic acid, triglycine, N,N′-ethylenediaspartic acid (EDDS), ethylenedioxybis(o-phenylenenitrilo)tetraacetic acid (BAPTA), desferoxamine, etc., any of which may be suitably used as a secondary “antioxidant”. Further useful antioxidants include endogenous defense systems, such as cearuloplasmin, heamopexin, ferritin, haptoglobion, lactoferrin, transferrin, ubiquinol-10, and enzymatic antioxidants; the less complex molecules including but not limited to N-acetylcysteine, bilirubin, caffeic acid and its esters, beta-carotene, cinnamates, flavonoids, glutathione, mesna, tannins, thiohistidine derivatives, triazoles, uric acid; spice extracts; carnosic acid, carnosol, carsolic acid; rosmarinic acid, rosmaridiphenol; oat flour extracts, gentisic acid and phytic acid, steroid derivatives; tryptophan metabolites, and organochalcogenides.

The term “area per chain”, or Ac, means herein the average molecular area divided by number of hydrophobic (most often aliphatic) chains per molecule. Experimental Ac values are typically method and readout dependent.

The term “aryl” as part of an “ion” preferably contains from 6 to 16 (C₆₋₁₆), from 6 to 14 (C₆₋₁₄), from 6 to 12 (C₆₋₁₂), or from 6 to 10 (C₆₋₁₀) atoms. Preferred heteroaryls have typically 5 to 10 C-atoms. Furanyl, imidazolyl, isothiazolyl, isoxazolyl, oxazolyl, pyrrolyl, thiazolyl and thienyl are thus particularly preferred. Somewhat less preferred heteroaryls are pyrazinyl, pyrazolyl, pyrazolinyl, pyridyl, pyridazinyl, pyrimidinyl, thiadiazolyl and triazinyl. The heterocyclyl or heterocyclic groups particularly useful for the invention as parts of (preferred) ions have typically from 3 to 10, from 3 to 8, from 4 to 7, or from 5 to 6 ring atoms and may include azepinyl, dihydrofuryl, dihydropyranyl, dioxolanyl, dihydropyrazinyl, dihydropyridinyl, dihydropyrazolyl, dihydropyrimidinyl, dihydropyrrolyl, dioxolanyl, 1,4-dithianyl, furanonyl, furanyl, imidazolidinyl, imidazolinyl, imidazolyl, isothiazolidinyl, isothiazolyl, isoxazolidinyl, isoxazolyl, morpholinyl, oxazolidinonyl, oxazolidinyl, oxazolyl, oxiranyl, piperazinyl, piperidinyl, 4-piperidonyl, pyrimidinyl, pyrrolidinyl, pyrrolinyl, pyrrolyl, tetrahydrofuryl, tetrahydrofuranyl, tetrahydropyranyl, tetrahydrothienyl, tetrazolyl, thiadiazolyl, thiamorpholinyl, thiazolidinyl, thiazolyl, thienyl, triazinyl, triazolyl, & 1,3,5-trithianyl groups. Somewhat less preferred ion components include benzimidazolyl, benzindolyl, benzoisoxazolyl, benzisoxazinyl, benzodioxanyl, benzodioxolyl, benzofuranonyl, benzofuranyl, benzoxazinyl, benzoxazolyl, benzothiazolyl, indazolyl, indolinyl, indolizinyl, indolyl, isoquinolinyl, oxazolopyridinyl, phthalazinyl, pteridinyl, purinyl, pyridopyridinyl, quinazolinyl, quinolinyl, quinoxalinyl, quinuclidinyl, and tetrahydroisoquinolinyl.

The term “bilayer” or “amphipat bilayer” or “lipid bilayer” means a molecular arrangement in which two monolayers of amphipats adhere together in a tail-to-tail fashion with the hydrophilic “headgroups” facing the polar (typically aqueous) medium on either side. Any non-confined bilayer is consequently tension-free. In the absence of an interfering surface, a lipid bilayer typically forms a vesicular structure, most often of quasi-spherical (and typically large and thus locally quasi-planar) form and only locally or exceptionally of a more curved, e.g. tubular, form.

The term “cation” means herein a positively charged entity solvable in water. For purposes of the invention, organic cations involving at least one nitrogen atom are particularly attractive. This includes substituted ammonium ions, such as aliphatic and aromatic amines. The non-radioactive monovalent alkali metal cations (particularly sodium and potassium, less often rubidium and caesium and only exceptionally lithium) are also suitable for use in the disclosed compositions. The divalent alkaline earth cations (beryllium, magnesium, calcium, strontium, barium) or the divalent cations of transition elements (manganese, iron, cobalt, nickel, copper, zinc, silver, gold, cadmium and mercury) are also suitable for the described formulations. The trivalent cations of such elements (e.g. iron (III), cadmium (III), or aluminium) play a limited, if any, role in the invention, as they typically condense and thus decrease rather than increase the associated charged aggregates adaptability, which is undesirable. Such cations can charge-up an otherwise neutral aggregate surface, however, which may be useful.

Exemplary primary amines relevant for this invention are the lower-chain alkylamines methylamine or ethylamine, as well as ethanolamine (2-aminoethanol), the corresponding bi-functional lower chain alkanediamines, such as ethylene-diamine. Exemplary secondary alkylamines with relatively short side-chains include dimethylamine, methylethylamine, diethylamine and methylethanolamine or diethanolamine, methyldiethanolamine, ethyldiethanolamine, dimethylethanolamine, deanol, ethylethanolamine, diethylethanolamine, and 2-(2-diethylaminoethyloxy)ethanol. Exemplary ternary alkylamines like triethanolamine or related molecules having at least one hydroxyl-group include 2-[bis(2-hydroxyethyl)amino]ethane-1,1,1-triol, [bis(2-hydroxyethyl)amino]methanetriol, 2-[2-hydroxyethyl(hydroxymethyl)amino]ethanol, 1-[2-hydroxyethyl(methyl)amino]ethanol, 1-[1-hydroxyethyl(2-hydroxyethyl)amino]ethanol, 2-[ethyl(hydroxymethyl)amino]ethanol, 2-[ethyl(methyl)amino]ethanol.

(Quaternary) Amines with four side groups, which are always cationic, include, e.g., a NH₄⁺, alkyl-, alkylaryl- or poly(arylalkyl)phenyl-ammonium cation or its (poly)alkylene oxide adducts, or an amino-terminated (poly)alkylene oxide adduct. The suffix -onium or -anium identifies permanently cationic groups, as e.g. in phosphanium, sulphanium, silanium, arsanium, oxanium ions and their substituted derivatives. Examples of the latter include, but are not limited to, alkoxy-hydroxy-oxo-phosphanium ions, dimethoxy(oxo)phosphanium, the corresponding aryl and heteroaryl substitutes, etc.; furthermore the related sulphanium cations, the mixed sulphonyl-phosphanium and its (4-methylphenyl) [symbol] substitute, 3-morpholin-4-ylpropylsulphonyl-oxidanium as a non-limiting example for an oxanium.

All amino acids contemplated for use in the aggregates have at least two, and may have more, ionisable groups, giving rise to cationic, anionic, zwitterionic or polyionic species. Arginine, glycine, lysine are common and, for the purposes of this invention, potentially useful amino acid buffers. Further useful amino acids and numerous other relevant buffers are detailed in various published tables, which also provide buffer information such as the dissociation constant(s) values.

Non-cyclic amino-salts having at least one cationic group are particularly useful for purposes of the invention, especially if they can buffer a preparation. Potentially useful compounds include, but are not limited to the salts of bis-TRIS, tromethamine=TRIS, aminomethylpropanol, aminoglycol, 2-amino-3-methoxy-2-(methoxymethyl)propan-1-ol, 2-amino-3-methoxy-2-methylpropan-1-ol, 2-amino-2-ethylpropane-1,3-diol (AEPD), 2-(2-aminoethyl)-2-(hydroxymethyl)propane-1,3-diol, 2-aminoisobutanol, 4-amino-2-tert-butyl-2-methylbutane-1,3-diol, in increasing order of lipophilicity and, by and large, alkalinity; the related glycine, bicine, tricine and aspartic acid are potentially useful, carboxyl-group containing and relatively hydrophilic, and in certain pH range zwitterionic, buffers; Pipes and Tes exemplify related sulphonic acid based compounds; Hepes and Hepps, or ADA, with two N-atoms per molecule, possess an even greater number of titratable groups.

The non-aromatic cyclic amines particularly important for the invention have a 5-, 6-, or even 7-member ring. The former group comprises, but is not limited to pyrrolidine, pyrroline the aromatic pyrrole and imidazole but also includes the corresponding mono- and di-derivatives with the lower-alkyl side chains that can be similar or mixed, (as in methylimidazole, dimethylimidazole, ethylimidazole, diethylimidazole, and methylethylimidazole, etc.). The correspondingly structured oxo-, hydroxy, methoxy, or halido-derivatives, oxazoles, and thiazoles also useful according to the invention. The six-membered rings comprise, progressively acidic, piperidines piperazines, pyridazines (1-N,2-N), pyrimidines (1-N,3-N) and pyrazines (1-N,4-N), any of which can also be mono-, di-, and trialkylated, e.g., as in mono-, di- and trimethylpyridines (i.e. picolines, lutidines, and collidines). In addition/alternative any such ring can have oxo-, hydroxy-, or methoxy-groups or halide atoms incorporated into any atom that will provide a stable compound, such as, e.g. orocic acid. Another alternative is to introduce a titratable N-atom on a side chain, as in (sulphinoamino)-cyclohexane.

Illustrative but non-limiting examples for piperazine-based salts include the molecule itself and its alkylated derivatives. Illustrative examples of pyrrolidino-group in the invention include, e.g. 3-pyrrolidinopropylamine1-(2-pyrrolidinylmethyl)pyrrolidine and 1-(1-pyrrolidinylmethyl)pyrrolidine, N-(3-pyridylmethyl)pyrrolidine, 3-(1H-pyrrol-1-ylmethyl)pyridine, etc. Azepin ions exemplify some of the cationic 7-member ring structures.

Additional cationic aromatic amines for suitable use in the invention include but are not limited to aniline, lower-alkylaniline, cyclohexylamine or its mono and dialkylates, such as N-methyl-, N-ethyl-, or N,N-dimethyl-cyclohexylamine, dimethylaniline, trimethylaniline, hydroxyaniline), dihydroxi- and trihydroxyaniline, 3-, 4-, 5- or 6-aminophenol), hydroxyalkylaniline, (di)halido-N-hydroxyaniline, N-hydroxyaniline), anisidine, N,N-dimethylaniline, benzylamine, benzylethanamine, benzylpropanamine, benethamine and the corresponding two N-atoms carrying benzathine and morpholinoalkanol, such as morpholinoethanol and morpholinopropanol in addition to epolamine.

The 6- and 5-membered ring combinations, e.g. in the relatively hydrophobic indoles, can be derivatised further, e.g. to 2-methylindole. In contrast, benzimidazoles carry 2 N-atoms in the 5-membered ring combined with a 6 membered ring. Acetyltryptophan carries one N atom in the 5-6 ring combination and one in the side chain; diaminonaphthalenes with two N-atoms have broadly similar properties as the former. By contrast, naphthylamines, having only one nitrogen in two 6-member rings, are more acidic and more hydrophobic. Even more water-adverse are the bisdiaminonaphthalenes, which are less preferred for use in the invention. The same holds true for benzylpiperazine.

The term “co-solvent” herein includes but is not limited to the group of short- to medium chains alcohols, such as C1-C8 alcohols, e.g. ethanol, glycols such as glycerol, propylene glycol, 1,3-butylene glycol, dipropylene glycol or polyethylene glycols, preferably comprising ethylene oxide units in the range from about 4 to about 16, e.g., from about 8 to about 12.

The term “Debye screening length” reflects the range of electrostatic interactions in an electrolyte solution. For example, in a 0.1 M monovalent salt solution, this has the value of 0.97 nm, which decreases or increases with the square root of an increasing or decreasing salt concentration, respectively.

The term “fragrance” means herein any pharmaceutically acceptable compound which, if incorporated into an embodiment, assists in masking and/or improving an odor of the formulation. Examples include but are not limited to linalool, menthol, cis-3-hexene-1-ol, geraniol, nerol, citronellol, myrcene and myrcenol, nerolido, benzaldehyde, eugenol, 1-hexanolhexyl acetate or dihydrojasmone.

The term “ion” refers to an anion or a cation, with one, two three, four, and potentially more, negative or positive net charges, respectively. Molecules having an unequal number of positive and negative charges may also be ions for purposes of the invention. “Ionic”, “anionic”, “cationic”, etc. have the corresponding meaning.

An uncharged but ionisable compound becomes charged during acid-base titration. The so-called pK then corresponds to the pH at which 50% of the studied titratable groups are charged due to deprotonation of acidic or protonation of basic groups, and is commonly known in the art. If not, then the pK value can be easily calculated (e.g. using SPARC), or simply measured. Molecular association, e.g. binding to an aggregate, changes the negative decadic logarithm of the corresponding dissociation constant in the bulk, i.e. pK, to a dissociation constant in an associate/aggregate, i.e. pK_{a,ass}/pK_{a,mem}, which can be higher or lower than the intrinsic pK, for reasons well known to the skilled person. pK_{a,ass}/pK_{a,mem} is also more sensitive to ambient conditions than pK for similar reasons.

The term “HLB” refers to the Hydrophilic-Lipophilic Balance number and the commonly used Griffith-nomenclature, which is also used herein and ranges between 0 and 20. Amphipat polarity, and thus hydrophilicity, increases with increasing HLB, and vice versa. Amphipats with a high HLB number consequently readily disperse/form micelles in water, and support oil-in water emulsions (o/w) formations. Amphipats with a low HLB number conversely tend toward water-in-oil (w/o) emulsions or minimally hydrated inverse or lamellar phase formations, if they hydrate at all. HLB calculations have been described (e.g., Pasquali et al., 2008, Int J Pharma 356: 44). The relationship between HLB and Ac has been described (Cevc, 2012, J Contr Rel, http://dx.doi.org/10.1016/j.jconrel.2012.01.005).

The HLB of many common surfactants is tabulated (“Handbook of Pharmaceutical Excipients”; “Handbook of Detergents, Part A: Properties”, G. Broze, Ed., Marcel Dekker, New York, 1999; “Handbook of Industrial Surfactants”, M. Ash & I. Ash, Synapse Information Res., 2008 [4th edt., with trade name references, components cross-reference, and lists of suppliers]. Another source, especially for establishing inter-substance correlations, is “Gardner's commercially important chemicals: synonyms, trade names, and properties”, G. W. A. Milne, ed., Wiley, New York, 2005.

The term “humectant”, or moisturiser, means herein a compound that at least helps maintain and ideally improves hydration, e.g. of the skin. Examples include but are not limited to glycerol, propylene glycol and glycerol triacetate, butylene glycol, other polyols (such as sorbitol, xylitol and maltitol, and polydextrose), acetamide and lactamide, natural extracts (e.g. quillaia), alpha-hydroxy acids (such as lactic acid), hyaluronic acid, pyrrolidine carboxylic acid (5-oxo-DL-proline, pyroglutamate), biphosphate, hexamethaphosphate, (tri)polyphosphates, sucrose, trehalose, and urea or their pharmacologically acceptable salts and derivatives (such as lower-alkyl-sorbates or polyoxyethylenes, alkylated, e.g. butylated, polyoxymethylene urea, etc), and ectoin.

The term “hydroxy” in the framework of this application means a hydroxy group on a fatty acid, unless specified otherwise. Chain-lengths for the preferred hydroxy-fatty acids vary from about C10 to about C30, more preferred from about C12 to about C22, and even more preferred from about C12 to about C20. Such fatty acids are normally saturated but can also be monoenoic.

The term “lipid” means herein a substance with at least partially fat-like characteristics. Each lipid of the invention thus has at least one extended lipophilic (i.e. hydrophobic and fat- rather than water-soluble, apolar) group, called the “chain” or “tail” (which is often but not necessarily linear). A lipid may moreover contain at least one hydrophilic (i.e. lipophobic and more water- than fat-soluble, polar) part known as the “headgroup”. A simple lipid can be represented with the following formula: Xₖ—Yₗ—Zₘ wherein at least one of the three counting-indices (k, l, m) is non-zero. The other two indices can then be positive or zero.

The term “membrane” is herein synonymously with the terms “bilayer” or “lipid bilayer”, unless specified otherwise.

The term “molecular area” means the average area occupied by a molecule in a locally flat molecular aggregate such as a monolayer at the air-water or air-oil interface, a vesicle bilayer, a stack of quasi-planar bilayers, or a lamellar phase. Molecular heterogeneity (e.g. headgroups or tails distribution within the studied molecular class) can preclude a molecular area definition at single molecule level. Even for a pure and well-defined substance, however, the measured molecular area is nearly constant only in a crystalline phase. The reported or independently determined areas for the fluid-crystalline (e.g. (quasi)lamellar L-alpha phase) differ by up to 25%, and occasionally more, due to various molecular area definitions and experimental choices. Where the Ac comparison relies on similar definitions and experimental methods, the result becomes reasonably constant and practically useful.

The term “NSAID” for the purposes of this invention refers to a compound commonly recognised to be a non-steroidal anti-inflammatory drug, or class of drugs imparting an analgesic, antipyretic and/or anti-inflammatory effects. Such compounds typically act as non-selective inhibitors of the enzyme cyclooxygenase, e.g. the cyclooxygenase-1 (COX-1) and cyclooxygenase-2 (COX-2) isoenzymes and include, but are not limited to substituted phenylacetic acids or 2-phenylpropionic acids, such as alclofenac, ibufenac, ibuprofen, clindanac, fenclorac, ketoprofen, fenoprofen, indoprofen, fenclofenac, diclofenac, flurbiprofen, pirprofen, naproxen, benoxaprofen, carprofen or cicloprofen; heteroarylacetic acids or 2-heteroarylpropionic acids having a 2-indol-3-yl or pyrrol-2-yl radical, such as indomethacin, oxmetacin, intrazol, acemetazin, cinmetacin, zomepirac, tolmetin, colpirac, tiaprofenic acid; indenylacetic acids, such as sulindac; heteroaryloxyacetic acids, such as benzadac; piroxicam, droxicam, meloxicam, tenoxicam, isoxicam, lornoxicam, ampiroxicam, cinnoxicam, sudoxicam, pivoxicam, tenoxicam, meclofenamate; acetaminofene (paracetamol); acetylsalicylic acid and its derivatives; diflunisal; etodolac; floctafenine; ketorolac; mefenamic acid; nabumetone; oxyphenbutazone; phenylbutazone; and salsalate.

The term “oil” means herein, first, the group of fatty acid esters of polyols, such as liquid triglycerides from natural sources, including but not limited to avocado oil, bergamot oil, borage oil, cade oil, Camelina sativa oil, caraway oil, castor beans oil, cinnamon oil, coconut oil, corn oil, cotton or grape seeds oil, evening primrose oil, hazelnut oil, hyssop oil, jojoba oil, linseed oil and marrow oil; Moringa concanensis as well as meadowfoam oil; olive oil; palm kernel, peanut primula and pumpkin oil; rapeseed or canola oil; saffron (safflower), sesame, soybean and sunflower oil; sea buckthorn oil and various fish oils, chicken fat, purcellin oil and tallow; plant and animal oils of formula R₉—COOR₁₀, in which R₉ is chosen from fatty acid residues comprising from 7 to 29 C-atoms and R₁₀ is an aliphatic chain comprising from 3 to 30 C-atoms, such as alkyl and alkenyl, e.g.; glyceryl tricaprocaprylate; a natural and synthetic essential oil, such as, e.g., eucalyptus oil, lavandin oil, lavender oil, vetiver oil, Litsea cubeba oil, lemon oil, sandalwood oil, rosemary oil, camomile oil, savory oil, nutmeg oil, orange oil, geraniol oil, and synthetic oils as defined below.

Second, the term oil can refer to a mineral or synthetic oil. The former group includes, e.g., alkanes ranging from octane to hexadecane, and liquid paraffin. Synthetic oils include fluorinated oils (e.g. fluoroamines, including but not limited to perfluorotributylamine), fluorohydrocarbons (e.g. perfluorodecahydronaphthalene), fluoroesters and fluoroethers; lipophilic esters of at least one mineral acid and of at least one alcohol; liquid carboxylic acid esters. The synthetic oils suitable for the invention may be chosen, e.g., from polyolefins, such as poly-a-olefins, e.g. poly-a-olefins from the classes of hydrogenated and nonhydrogenated polybutene poly-a-olefins, such as hydrogenated and non-hydrogenated polyisobutene poly-a-olefins.

A third group of oils suitable for purposes of this invention are volatile and non-volatile silicone oils, which can be combined with oil(s) lacking silicium atoms. When used, the total amount of silicone oils generally ranges, e.g., from 1% to 50% by weight relative to total weight of oils.

The term “partition-ratio” (P) herein means the ratio of concentrations of a compound in (or on) the inventive aggregate and in the (typically aqueous) suspension medium at equilibrium. Information about (and semi-quantitatively comparable) water-octanol partition-ratio of many chemicals can be derived from/through publicly available databanks, or alternatively measured in a conventional fashion. Partition-ratio changes with molecular ionisation, often increasing between 1±0.75 and 2.5±0.75 log-units per net charge added in a 0.1 M 1:1 salt solution. More hydrophilic ions experience smaller changes than the less hydrophilic ions. The skilled person can use the conventional distribution ratio concept to assess molecular suitability for purposes of the invention, based on the guidance provided herein, which takes into account any molecular ionisation (note that partition-ratio of neutral compound Pₘₑₘ^{N} always exceeds the corresponding charged compound partition-ratio Pₘₑₘ^{I}). One recently published method can be used to improve the accuracy of such analysis (Elsayed et al., 2009, Pharm. Res. 26, 1332).

The term “pharmaceutical agent”, which is herein also referred to as a “drug” or (pharmacologically) “active ingredient”, means any pharmaceutically active substance approved and/or registered with a competent authority for use in or on mammals, especially humans and companion animals. Such agents may include drugs for treating the gastrointestinal tract (digestive system), the cardiovascular system, the central nervous system, pain and consciousness (analgesic drugs), musculo-skeletal disorders, the eye, the ear, nose and oropharynx, the respiratory system, endocrine problems, the reproductive system or urinary system, contraception, obstetrics and gynecology, the skin, infections and infestations, the immune system, allergic disorders, nutritional purposes, neoplastic disorders, or diagnostic purposes. For further info see e.g., Dictionary of Pharmacological Agents, C. R. Ganellin; D. J. Triggle; F. Macdonald, CRC Press, 1996.

The term “phase diagram” herein means a ternary, or pseudo-ternary, quarternary or pseudo-quaternary, and rarely quinternary phase diagram. Typically, such a phase diagram pertains to one temperature, which is not a must. If no suitable phase diagram is available, a person skilled in the art will know how to construct one using standard laboratory procedures including, but not limited to polarizing microscopy, spectroscopic, and in rare cases, scattering methods. To generate a phase diagram, it may suffice to inspect preparations optically (if necessary, under a microscope) after proper equilibration, which can be accelerated by transient heating, stirring, or centrifugation.

The term “polarity unit” number, or nP, defines herein the number of at least partially hydrophilic repetitive units, typically within the polymeric polar headgroup of an amphipat, which corresponds to one oxyethylene unit in the polar headgroup attached to a linear-chain polyoxyethylene (=PEG)-fatty-ether. Amphipats of the formula:

R′—(O—CH₂—CH₂)ₙ—OR″

herein have thus, by definition, “n” polarity units in each head when R″ is a hydrogen atom; a fatty alcohol consequently carries no polarity unit, i.e., has nP=0. Each carbonyl group or nitrogen atom at the headgroup attachment site(s) reduces nominal polarity units count by around −0.5. Each oxypropylene segment corresponds to around ⅓ polarity units. Each oxyethylene (=EO) or oxypropylene (=EP) segment attached stochastically to a sorbitan-ring that is also coupled to at least one fatty residue contributes effectively 0.59/n polarity units to the headgroup attached to n hydrophobic chains. This observation is thus applicable, for example, to the amphipats having the formula:

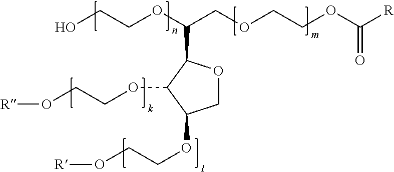

By way of example, Tween 80 (with nEO=20 and R═C18:1, R′═R″=protons, i.e. n=1) has a similarly polar headgroup as a linear PEG-ether with the same hydrophobic chain length and nEO˜11.8; Tween 85 (with nEO=20 and R═R′═R′═C18:1, i.e. n=3) roughly corresponds to a linear PEG-ether with nEO˜3.9 and C18:1. Neglecting the possible sugar stereochemistry effects, a mono-aliphatic hexose-ester or -amide carries around 3.8 polarity units. Most commercial sugar-derivatives have n>1 hydrophobic chains attached to each sugar residue, however. This affects the resulting amphipat polarity, which is then “distributed over” n chains, giving nP˜3.8/n as the effective polarity units count. The second sugar segment in a headgroup (as in maltose vs. glucose) typically increases the effective polarity units number by around 10-20%, dependent on the sugar type. A polyglyceride polarity units number is also sensitive to distribution and total number of hydrophobic chains on each headgroup and ranges from around 1.65 for an essentially linear mono-aliphatic-oligo- or -polyglyceride through 0.8 down to around 0.2 polarity units per C18:1 hydrocarbon chain in a stochastic oligo-fatty-ester-oligo- or -polyglyceride. (A commercial fatty-pentaglyceride thus can correspond to a PEG-fatty-ether with nEO˜3 and its nominally similar kin from a different manufacturer to a PEG-fatty-ether with nEO˜0.3.) N,N-dimethylamine-N-oxide corresponds to around 5 polar units. A glycerophosphocholine or a charged, but electrostatically screened, glycerophosphoglycerol on a double-chain lipid correspond to around 2 polarity units per fatty chain and to around 4.5 units per hydrocarbon chain of the corresponding lysophospholipid. A double-chain glycerophosphate-monomethyl-ester or glycerol-phosphoethanolamine-(N,N)-dimethyl can carry around 1.4 polarity units per fluid fatty chain each. The corresponding mono-charged, but screened, phosphatidic acid contributes zero polarity units to a bilayer, which is thus controlled only by chains. Exchange of a phosphate headgroup on an amphipat with a sulphate group does not appreciably affect molecular polarity. Based on these values, one will be able to assign polarity unit equivalents to the other relevant headgroups following a consultation with the published, or otherwise readily obtainable, information.

The term “practically acceptable” means herein that a compound, analytical or manufacturing process, packaging or utilisation form is deemed to be acceptable for or by any competent regulatory body or the designated customer.

The term “preferred chain(s)” means herein one or more acyl, alkyl, alkenyl, alkynyl or alkenoyl hydrocarbon radical(s) with C8 to C24, more preferred with C12 to C22, even more preferably with around C16 to around C20 and most preferably with around 18 C-atoms per chain. When constructing a non-invasive drug carrier according to the invention, any preferred chain should be fluid at least at body surface temperature (i.e. typically around 30-32° C. and more broadly between 25° C. and 37° C.), with chain fluidity above 0° C. being desirable. Exemplary hydrophobic chains meeting this goal are short saturated chains with about 8 to about 14 and preferably about 10 to about 12 C-atoms per chain. Another preferred type of chain is longer straight chains that are fluid in the target temperature ranges by double bonds or side groups (as in the branched alkenoyl, alkoxy or polyoxy-alkylene hydrocarbon radicals). The preferred chains from the latter group have typically around C12 to around C22, preferably around C14 to around C20, more preferably around C16 to around and C18, and most preferably around 18 C-atoms. Alkenoyls having 1-3 double bonds per chain are preferable, the lowest number ensuring fluidity being preferred. The cis-conformation is more desirable than a trans-conformation of the double bond. Simple alkoxy-alkylenes are preferred over polyoxy-alkylenes and chain modification in the middle or the upper part of the hydrocarbon radical is more preferable than modifications occurring in the end regions of the chains.

A non-exhaustive listing of preferred chains includes relatively short chains, in particular the dodecanoic or lauric chains, and also tetradecanoic or myristic, decanoic or capric, and in some instances octanoic or caprylic and tridecanoic chains. Preferred mono-unsaturated oligo-alkenoyls with C18 per radical include, but are not limited to cis-6-octadecenoic or petroselinic, cis-9-octadecenoic or oleic, and cis-11-octadecenoic or vaccenic, plus the nearly as preferred di-unsaturated 9-cis, 12-cis-octadecadienoic or linoleic or gamma-linoleic, 12-cis,15-cis-octadecadienoic or alpha-linoleic chains. The preferable longer mono-alkenoyls are mainly of the mono-unsaturated gondoic or 11-cis,14-cis-eicosadienoic kind. Useful but less stable against oxidation, and thus less preferred, are the tri-unsaturated alpha- or gamma-linolenic and di-homo-gamma-linolenic chains; it may thus be preferable to use 15-hydroxy-hexadecanoic and 17-hydroxy-octadecanoic or ricinoleic, or iso-staric, iso-palmitic, or iso-myristic chains instead.

A further group of hydrophobic “chains” preferred for the purposes of the invention encompasses cycloalkyl, aryl, C7-C14 aralkyl, heteroaryl, or heterocyclyl derivatives having a similar total number of C-atoms per radical as specified for non-cyclic compounds. Hydrophobic chain(s) attached to a polar headgroup with a bond that is not an ester or ether, as in sphingo- or thio-lipids, should also preferably contain a similar number of carbon bonds in like fashion. A preferred chain may also contain Si-atoms, i.e. be a silane with physical properties sufficiently similar to those of the specified preferred hydrocarbon chains. Preparations designed for the noninvasive delivery of pharmaceutical agents can benefit from using relatively short, around C12, chains or from chain branching; both options effectively create two tails with 4 to 14 and preferably no less than 4 and no more than 12 C-atoms per segment/branch. Such selection should ensure both bilayer fluidity and a desirable minimized bilayer thickness that does not sacrifice physical stability of the vesicle.

The term “preferred ion” means herein the charged form of any pharmacologically or practically acceptable compound with partition-ratio, log P, and more preferred, a partition ratio, i.e. log P, of around −1±3 (or alternatively, a partition-ratio log P=−1±2.5 or log P=−1±2 for the neutral compound form), under the proviso that at least 20% of the underlying salt is ionised, more preferably at least 33%, even more preferably at least 50%, and most preferably more than 66%, unless the “ion” is a zwitterion. This requirement is sensitive to preferred ion pK and pKa (or pK_{a,ass}), the chosen preparation pH, the combined amphipats concentration, and the selected total salt concentration.

The term “range”, when used in conjunction with ≧2 numerical values, means that the numerical value can be any value in said range. For the purposes of this invention, it also means that within the broadest range specified, any narrower range can be chosen using 50%, 33%, 25%, 22.5%, 20%, 17.5%, 15%, 12.5%, 10%, 9%, 8%, 7%, 6%, 5%, 4%, 3%, 2%, or 1% of the entire range. By way of example, a range of 1 to 10 can thus be subdivided and/or limited to 1 to 2, 2 to 3, 3 to 4, 4 to 5, 5 to 6, 6 to 7, 7 to 8, 8 to 9 and 9 to 10 or else to 1 to 3.33, 3.33 to 6.66 and 6.66 to 9.99 or 3.33 to 9.99, or from 1 to 4, 4 to 7, 7 to 10, 1 to 7 or 4 to 10; or else from 1 to 3.25, from 3.25 to 5.5, from 5.5 to 7.75, from 7.75 to 10, from 1 to 5.5, from 1 to 7.5, from 3.25 to 7.5 from 3.75 to 10, or from 5.5 to 10.

The term “salt” herein is used as a synonym for the term “simple or complex, organic or inorganic salt”, unless otherwise specified.

The term “silicone” herein means a linear and cyclic, branched and crosslinked organosilicon polymer and organosilicon oligomer of variable molecular weight, obtained by polymerisation and/or polycondensation of suitably functionalised silanes, and which comprise repeating units in which the silicon atoms are connected together by oxygen atoms (siloxane bond≡Si—O—Si≡) and are optionally substituted with at least one hydrocarbon-based group, which is bonded by way of a carbon atom of said hydrocarbon-based group to said silicon atoms. Typical hydrocarbon based groups include alkyl groups, such as C1-C10 alkyl groups and methyl, fluoroalkyl groups, aryl groups, such as phenyl, and alkenyl groups like vinyl; other groups that can be bonded, either directly or by way of a hydrocarbon-based linking group, to the siloxane chain encompassing a hydrogen atom; halogens such as chlorine, bromine and fluorine; various thiols, alkoxy groups, polyoxyalkylene groups, such as polyoxyethylene and polyoxypropylene, polyether groups, hydroxyl, hydroxyalkyl groups, amide groups, acyloxy groups, acyloxyalkyl groups, amphoteric groups, betaine groups, anionic groups (so-called “organomodified” silicones).

The term “simple or complex, organic or inorganic salt” means a simple or complex, organic or inorganic anion or a simple or complex, organic or inorganic cation or a combination thereof.

The term “sufficient” used in the context of deformability, stability, or efficiency tests means that the test result falls within ±50%, preferably within ±33%, more preferably within ±25%, and ideally within ±20% limits.

A person skilled in the art may prepare equivalents to the specific formulations and procedures according to the teachings of the present invention. Such equivalents therefore fall within the contemplated scope of the invention and are consequently encompassed by the claims. The contents of all cited references, patents, and patent applications are hereby incorporated by reference. The appropriate components, processes, and methods of these cited disclosures may be suitably selected for use in the embodiments of the present invention.

The term “vesicularisation time” refers to the time required to transform an originally opaque suspension (i.e. having an optical density >>3) to an opalescent/transparent suspension with a much lower optical density using external stress, e.g. generated with an ultrasound transducer, high-shear homogeniser (Ultra-Turrax®, IKA) or rotor-stator homogeniser. For comparative purposes, the final optical density can be chosen arbitrarily, so long as it is at least 3-4-times lower than the starting optical density, and the compared suspensions are tested under similar conditions in terms of total amphipat concentration, temperature, total volume, etc. Transformation into small vesicles can be identified, roughly, with the final optical density of a non-absorbing sample around 0.8±0.4 (1 cm light-path; 800 nm incident light wavelength).

### 5.1. Amphipat Aggregation

Amphipats normally have a tendency to aggregate in polar media to balance out intermolecular as well as inter-aggregate forces. Any closed (mixed) amphipat bilayer is thus tension free, at least on average. Stress fluctuations near aggregates in a suspension can prompt local composition or form adjustments that (re)establish the original tension freedom. Adaptable aggregates known in the art achieve this balance via a partial (de)mixing of the bilayer-forming amphipats, i.e. mainly by changing lateral distribution of lipophilic molecules within the bilayer, which thus becomes more deformable. Without being bound to any one theory, the aggregates according to the present invention exploit hydrophilic additives to a similar end, being compatible with a greater and more complex variety of amphipats, not just the known and predominantly if not exclusively phospholipid-based combinations. It was thus surprisingly found that the right, situation-specific choice of such additive(s) and its/their correct quantity is the key to a timely additive (re)distribution near suspended aggregates, mainly perpendicular to the vesicle surface. The resulting, mainly transverse, redistribution of essentially hydrophilic molecules near the aggregate bilayer enhances or replaces lipophilic componets redistribution making the bilayer (more) deformable, and the vesicle thus (more) adaptable, e.g. under influence of charge-charge interactions. The outcome is advantageous in several aspects: it permits the ready passage of potentially large aggregates through an otherwise impenetrable barrier, such as the skin, and ensures (a mixture of) amphipats to be more readily dispersible and/or more stable and/or better suited to carry cargo.

Any amphipat having a sufficiently prominent hydrophobic segment causing formation of an aggregate that is not merely an oligomer can be a lipid in the context of the present invention. To characterise and differentiate between potentially useful lipids, it is advantageous to distribute them into three classes:

ML: monolayer/micellar phase/(quasi)isotropic watery “bulk phase” formers;

BL: bilayer vesicle and/or lamellar phase formers; and

IM: inverse-micellar and (quasi)isotropic oily bulk phase formers.

Preparations of the invention are typically made from molecules belonging to the former two classes mixed in proportions that yield an average area per chain, Ac, in the range 0.35-0.55 nm² approximately, which is proximal to the lower ML-class limit in a broad sense. BL-class molecules typically occupy an area of 0.18-0.22≦Ac/nm²≦0.35-0.55 within a bilayer. IM-class molecules predominantly have an Ac≦0.18-0.22 nm² (for the gel phase with untilted chains) and up to around 0.28 nm² (for chains in a fluid bilayer).

### 5.2 Aggregate Sensitivity to the Suspending Medium

Most BL-type amphipats are either non-ionic or zwitterionic, and occasionally amphoteric. Many s ML-class amphipats are conversely charged, like many types of active ingredients, which alone or together yield charged aggregates. The latter are consequently pH- and ion-sensitive.

Fatty acids with more than 6 carbons per chain are normally uncharged at pH<pKa˜6.5-9.5 and are then of the IM-type. Their anionic counterparts, fatty soaps, which prevail at pH>pK_{a,ass}, fall into the ML-category, unless they are of the BL-type (which is normally the case with fatty acids with very long and/or branched chains). Likewise, the natural gall-acids are uncharged and insoluble at low pH, but form relatively soluble bile salt ions at pH>pK_{a,ass}˜6.5-8.5 dependent on the total amphipat and salt concentration. It is therefore important to consider, or perhaps exclude, ionisation changes when preparing formulations according to the invention.

An ion interacting with an aggregate of the invention may affect the aggregate properties, including adaptability, after ion-aggregate association. Considerably lipophilic and rigid, aggregate-adsorbed ions (e.g. the preferred ions with at least one relatively long aliphatic, aryl or heteroaryl segment) are prone to decrease bilayer flexibility and permeability, and thus lower bilayer deformability and thee resulting aggregate adaptability relative to ion-free bilayers. Conversely, considerably lipophilic, aggregate-adsorbed ions with flexible, and optionally polar, side-chains are likely to increase bilayer flexibility, and thus improve aggregate relative adaptability. (Overly hydrophilic ions with equally flexible side-chains tend to enhance aggregate adaptability less if their interaction with, or attraction to, a charged aggregate surface is too weak). Both phenomena can be practically valuable. The first may be applied to suppress aggregate solubilisation by the bilayer destabilising agents, and the latter can be applied to ensure sufficient aggregate adaptability in the opposite situation.

Given the choice, when aiming to modulate aggregate adaptability and stability, a suitably hydrophilic additive (as reflected in its distribution ratio) should be employed in the formulation. For cationic or neutral surfaces, the preferred additive will thus be an anion, and for anionic or neutral surfaces a cation. For an originally neutral aggregate surface the preferred anion or cation will be chosen bearing in mind that the aggregates comprised of uncharged/non-ionic amphipats can gain charges through adsorption of molecules from the surrounding solution, such as charged drugs or charged excipients. In addition, ions adsorbing to (or at least accumulated near) an aggregate surface are preferred, with the selected ion being mostly mono-charged. The likelihood of any resulting problems decreases with decreasing Debye's length. In turn, a shorter Debye's length diminishes the preferred ions accumulation near an aggregate surface. The optimum formulation may therefore have to be found in an iterative procedure, using one of the tests described herein, to determine the practically best concentration of the selected ion(s).

Well-established and published experimental methods are available to determine molecular ionisation and to test ion relative concentration at the aggregate surface and in the bulk, if necessary. Classical acid-base titration, zeta potential measurement, testing charged-label binding to aggregate surface, studying ion effects on an aggregate surface (such as adaptability and/or colloid stability) can all serve this purpose. Such methods can also confirm that the preferred ion-aggregate interaction does not significantly and adversely change the constituent dissociation constant(s).

In some embodiments of the invention, ionisable groups RPO₄²⁻ or RSO₃⁻ in phospho- or sulpholipids; COO⁻ in surfactants of the following formulae:

HOOC—CHR—CH₂—COO-Eₙ

HOOC—CHR—CH₂—COO-Eₙ-O—CO—CH₂—CHR′—COOH; or

R₁R₂R₃N⁺ in amino-surfactants compatible with compounds of formulae:

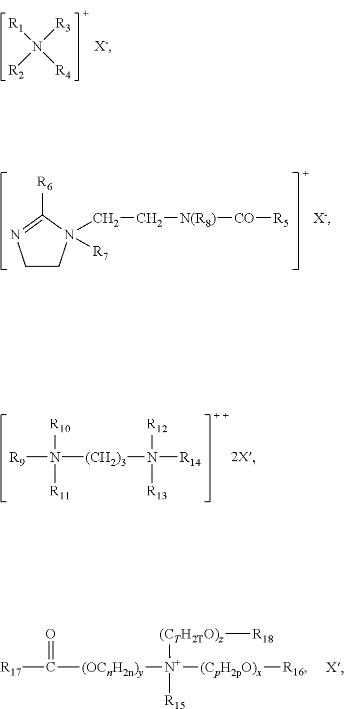

wherein R₁, R₂, R₃, and R₄ may be identical or different and are either aliphatic groups comprising from 1 to 30 C-atoms and/or aromatic groups, such as aryl and alkylaryl groups; or R₁R₂R₃R₄P⁺ or R₁R₂R₃S⁺ in phosphonium and sulphonium compounds, or any dissociable group on an active and/or some other ionisable ingredient may have to be used in at least partially charged form. Thus, one should use at least one buffer in the form of simple or complex, inorganic or organic salt and/or seek to gain buffering capacity from other excipients, such as polymeric ionic thickening agents, to gain control over charges. Optionally, at least one of the ions formed by such excipients dissolution/dissociation will be a preferred ion according to the invention. Even more preferable, the drug representing the adaptable aggregate's payload will be introduced into a preparation of the invention as the salt with the preferred ion.

For some embodiments, it may be convenient to include a spacer between the charged group and the aliphatic or aromatic chain or to separate several charged groups on a single molecule with a spacer. (In the latter situation the Debye's length is an important reference length.) Suitable spacers are, e.g., oligomers of oxyethylene (PEG 2 to around PEG 10 and more preferably PEG 2 to around PEG 6 or even PEG 2 to PEG 4), similarly long oligomers of oxypropylene (i.e. PPG 2 to PPG 10, or PPG 2 to PPG 6, etc) or short aliphatic chains (optionally with incorporated halide atoms or side chains). For some embodiments, it may be useful to introduce sufficiently polar segments into the vicinity of an important charge, e.g. on the additive of the invention. Lower alkyl alcohols with one or more hydroxy-groups are also suitable radicals useful for the purpose.

### 5.3. Aggregate Compositions

Known aggregate compositions require relatively high concentrations of a surfactant, a surfactant-like drug, or of a drug and surfactant combination to obtain highly deformable bilayers that can effectively cross a semipermeable barrier. These compositions further require repeated and extensive lipid testing for successfully determining a practically useful starting aggregate composition. By contrast, the present invention overcomes these drawbacks by describing, in detail, how to preselect, and optimize, a target formulation by efficiently selecting aggregate components based on available component information according to the guidelines and/or selection tools described herein.

Suitable aggregates can be quasi-spherical to begin with. They are normally comprised of a non-lipidic—typically aqueous—core surrounded by a few, or even just one, bilayer(s). The bilayer(s) of the invention is(are) normally composed of sufficiently different amphipats to allow bilayer re-arrangement during aggregate shape transformation, e.g. via lateral and (often pore facilitated) transbilayer amphipat motion. Amphipat chain fluidity is helpful, if not necessary, for molecular rearrangement as well as aggregate deformation. Aggregate stability, as a further prerequisite for proper functioning of the inventive aggregates, is supported by moderate bilayer fluctuations, which are also facilitated by chains fluidity. Using ions rather than surfactants to modulate such fluctuations can therefore keep the latter better at bay

### 5.3.1 Amphipathic Components

When producing the aggregates of the present invention, one may conveniently start by scrutinising the area per chain (Ac) of each putative amphipathic ingredient of the formulation. This allows a simple calculation, e.g., based on the assumed relative molecular concentrations needed to attain the desired average area per chain. Ideally, the latter should be between around 0.35 nm² and around 0.55 nm², and is often advantageously around 0.45 nm² for C18 chains and around 0.42 nm² for C12 chains. In either situation, the Ac should be preferably as close to the upper BL stability limit as the preparation stability permits, after accounting for experimental values uncertainty, pH changes, ion binding, and concentration effects in the bulk, at the aggregate surface, and at the site of final application. The calculation should moreover optimally include a term allowing for (preferred) ion effects on aggregates of the invention (e.g. by postulating a linear correlation between the adsorbed preferred ion quantity and the studied aggregate characteristic).

The resulting starting experimental formulation is then tested for adaptability and stability, as described elsewhere herein. Alternatively, one may begin by testing several sensible additives, such as one or more preferred ions and/or one or more preferred ion concentrations. The results analysis reveals the relative benefits or detriments of tested additives/ions for purposes of the invention. The final formulation is then adjusted correspondingly. A related simpler, but potentially less precise, preselection relies on the calculated polarity units/HLB number of each putative aggregate builder.

When working with fairly compounds, including some of the amphipats useful for the invention, one must know their relative concentration in an aggregate, and calculate such concentration, if necessary, from the individual absolute concentration and distribution ratio. The concentration of (the preferred) ions, which can belong to most water-soluble ingredients of a formulation, is normally specified in absolute terms, since the preferred distribution ratio of the ions is often in the range of −3 to −1. If this ratio is higher and/or if an ion-aggregate association significantly depletes ions from the bulk, one should refer to the relative rather than absolute concentrations for such ions. Relative concentrations are alternatively expressible as mol-per-mol (=mol/mol or mol:mol) or as wt.-per-wt. ratio. In the ratio calculation, each compound associated with an aggregate bilayer should be accounted for.

### 5.3.2 Additives

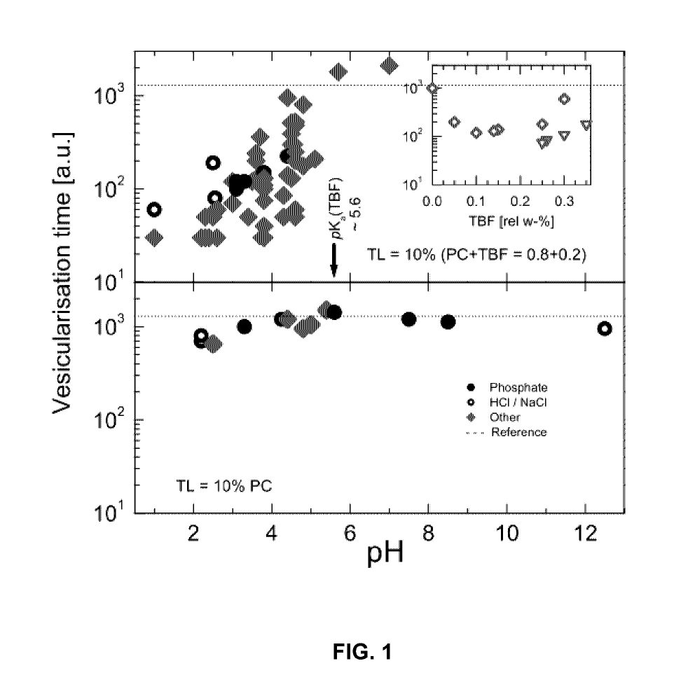

Some embodiments of the invention provide aggregate compositions that might appear to resemble known formulations. However, the surprising discovery of this invention is that by replacing a portion of the otherwise necessary excipients or surfactants of the known compositions with the described additives of the present invention, such as the preferred ions, the additives can act in combination with the chosen amphipats and unexpectedly function as surrogate excipients or surfactants within the aggregate, as the case may be. This typically significantly improves the performance of the aggregates by at least 20% compared with corresponding preparations with no additives of the invention included, i.e. the known preparations. Alternatively or additionally, the improved preparations herein can provide increased drug solubility and/or enhance the positive influence of a drug on aggregate adaptability and/or stability by at least 20%. FIG. 1 illustrates an exemplary improvement, namely the effect of buffer selection on vesicularisation time of the reference vesicles (i.e. containing >90% pure soybean phosphatidylcholine) compared to vesicles loaded with terbinafine as a function of the bulk pH.

Some embodiments of the invention thus relate to aggregates comprised of least two amphipats, one amphipat with less than 10 μM solubility in water and the other amphipat with an aqueous solubility at least 10 times higher, which together form adaptable aggregates that can advantageously cross pores much smaller than the aggregate's own diameter at least 20% more efficiently due to the inclusion of the described additives into the instant preparation. Some aggregate compositions are made from at least two amphipats, or from at least one commercial pluricomponent amphipat product, characterised by a HLB number in the range of 12-12.5>HLB>6.5-7.5, and preferably around HLB=10.5±2, such that upon dispersion in a polar fluid, e.g. water, form smaller aggregates at least 20% faster and/or aggregates at last 20% more capable of crossing pores narrower than their own diameter due to the inclusion of the described additives.

Some embodiments of the invention encompass aggregates prepared from at least one multiple-component amphipat or several different amphipats that, upon dispersion in an aqueous medium, are diminished in size at least 5 times, and preferably at least 10 times, easier or faster than a comparably concentrated preparation made of >90% pure phosphatidylcholine extracted from soybean, and/or pass through relatively narrow pores at least 20% easier/faster than such phosphaticylcholine aggregates without any of the described additives. The amphipats in some embodiments are selected to be nonionic and/or zwitterionic and/or amphoteric and can include nonionic amphipats having one or several hydrophilic segments per headgroup, which can be similar or different, and attached to at least one hydrophobic segment to ensure the amphipat(s) association with the aggregates according to the invention.

To ensure proper aggregation, the hydrophobic segment in the composition must have typically at least 8 and more often at least 10 C-atoms attached via an ester, ether, amide, sphingosine or thioester bond to the polar headgroup(s); any molecule with several hydrophobic anchors, or more than one polar segment per headgroup, can involve different such bond types, and then have a slightly higher total number of C-atoms in all chains taken together. The at least one hydrophilic segment may then be an acceptable polar group or its polymer, such as a lower, linear or branched, alkyl-chain alcohol hydroxylated on at least 50% of its C-atoms, or else an amine oxide, an 1-amino-1-sulphosulphanylalkane, an amino-alkane, a sulphonic or -sulphinic acid, betaine or sulphobetaine, a dimethyl-ammonio]-1-alkane-sulphonic, -phosphonic, or -acetic acid, an imino acid, a sugar (optionally comprising or attached to an N- or S-atom) or its lactone, a phospho-S,S-dimethyl mercapto short chain alkanol, a secondary or ternary sulpho- or sulphono-short chain (poly)alkanolamine (e.g. sulphocholine or -dimethylethanolamine), zwitterionic amino acid, or a glycerophospholipid headgroup.

After assigning the appropriate polar units number, nP, to each such polar headgroup, the headgroup length suitable for making preparations of the invention can be defined in terms of nP per hydrophobic segment with nC C-atoms that are at least partly fluid, and directly or indirectly attached to at least one hydrophilic headgroup. (When several hydrophobic units are used, n>1, the term “nC” refers to the average number of C-atoms per hydrophobic unit.) An amphipat with such a headgroup in preparations of the invention can optionally be supplemented with a smaller quantity of one or more further amphipat(s) having a similar or different, but typically more polar headgroup (i.e. in the case of a similar structure more extensive), such that on average the same preferred nP-criterion is fulfilled. The concentration of such further amphipat(s), if any, may then be chosen to be relatively lower if nP of the first amphipat headgroup is chosen to be higher, and vice versa. Each such headgroup should preferably have around 5nC/24 to around 8.5nC/24 polar units per hydrophobic segment. The nP is moreover preferably chosen so that the sum of all polar units on the employed amphipats is close to the upper polar units number limit specified above. However, for rather heterogeneous amphipat combinations an even higher polar units number may be tolerable (especially where greater diversity or longer chain length is present); the same holds true for the amphipats with relatively long hydrophobic chains, and/or for the amphipats with more than one hydrophobic chain per polar headgroup (i.e. n>1).

More specifically, the hydrophilic segment may be chosen, e.g., to be an oligomer or a polymer of a polyalcohol, such as ethyleneglycol, propyleneglycol, glycerol, butanetriol, pentanetriol or pentanetetraol, or a sugar. Such at least one hydrophobic segment in some embodiments of the invention has nC C-atoms attached via an ether bond directly or indirectly to the at least one hydrophilic headgroup comprising a chain of around 5 times nC divided by 24, i.e. 5nC/24, to around 8.5nC/24 oxyethylene units per hydrophobic segment. Such first amphipat may moreover be supplemented with a second amphipat with a similar or different, but typically more polar (i.e. in the case of a similar structure longer) headgroup. The concentration of the latter, if any, should be relatively lower if the repetitive (polar) units number in the first amphipat headgroup is chosen to be higher, and vice versa. In practice, this often means that the sum of all oxyethylene groups in the first and the optional second amphipat (if the latter is also a polyoxyethylene derivative) is between around 0.21nC and around 0.38nC per hydrophobic chain, relatively higher values being tolerable and preferred for more heterogeneous combinations. For the ester- or amide-bonded fatty-polyoxyethylene amphipats the corresponding values are somewhat higher, often by about 10-30%.

In some embodiments, in which the speed of formulation vesicularisation and/or the resulting aggregate adaptability is increased at least 20% by inclusion of additives of the invention, the at least one hydrophobic segment with nC C-atoms on each aggregate-forming amphipat is directly or indirectly attached to the at least one hydrophilic headgroup with around nPO=1 to around nPO=8 oxypropylene units per hydrophobic segment, which are further attached to a polyoxyethylene segment with nPO/3 fewer repetitive units per hydrophobic segment than in the corresponding oxypropylene-free amphipat. The rule of optionally supplementing the first with the second amphipat is otherwise similar as for the EO-based headgroups. (In case of such supplementation, the total number of hydrophilic segments in the first and in the optionally included second amphipat may be reduced by 1 for each 2-3 oxypropylene units included into amphipat headgroup; more heterogeneous headgroup combinations and longer disordered hydrophobic chains requiring and tolerating relatively higher sums.)

In yet another embodiment, additives of the invention are used to modulate vesicularisation and/or adaptability of aggregates formed from amphipats with n hydrophobic segments, each on the average comprised of nC C-atoms, acting together as hydrophobic anchors that are directly or indirectly attached to at least one hydrophilic headgroup based on a sorbitan ring with up to around 2nC/3n oxyethylene units (=EO) attached thereto. The first amphipat can also be supplemented similarly with another amphipat in such preparations. Total sum of all EO groups/hydrophobic chain in the first and the optional second, sorbitane containing, amphipat is in the resulting blend between about 3 and about 15, preferably between around 4 and around 13, and most preferably between about 5 and around 11 per C18 chain length and correspondingly less for the shorter chain lengths. Use of more heterogeneous headgroup combinations potentially requires up to around a 50% higher relative sum, i.e. use of more than 11 EO groups/hydrophobic chain. Shorter or less disordered hydrophobic chains typically require lower relative sum usage, i.e. rather around 5 than around 11 EO groups/hydrophobic chain.

Another embodiment of the invention concerns aggregates formed from the amphipats with n hydrophobic segments having nC C-atoms, on the average, which are attached directly or indirectly to between around (1+(n−1)^{0.55})(nC/12) and around (2+(n−1)^{0.55})(nC/12) glyceryl units in the case of quasi-linear molecules. Where this is the case, the first such amphipat type may be supplemented with a second amphipat being different or similar but typically more polar. (If such second amphipat has a similar headgroup, the headgroup will be longer.) The tolerable concentration of the second amphipat, if any, decreases with the first headgroup length, i.e., with the chosen number of repetitive units in the first amphipat headgroup and vice versa. In contrast, molecules having more stochastic hydrophobic chain distribution typically require higher number of glyceryl units per hydrophobic chain. (More specifically, the sum of all glyceryl groups in the first and optional second amphipat with similar headgroup type and different headgroup length may be between around nC(1.5+(n−1)^{0.55})/12 and around nC(2.5+(n−1)^{0.55})/12, more heterogeneous headgroup combinations and longer disordered hydrophobic chains potentially requiring around ⅔ higher and the stochastic chains attachment up to a 10 times higher total number of glyceryl units per hydrophobic chain.)

In another embodiment, additives of the invention are used to modulate properties of the inventive aggregates made of amphipats with at least one hydrophilic residue in a headgroup which is a sugar, and preferably a mono- or di-hexose or a mono- or di-heptose, attached directly or indirectly to between around 18/nC hydrophobic segments and around 40/nC hydrophobic, nC long segments. Such first amphipat may be supplemented with another amphipat (with preferably but not necessarily different headgroup); if used and more polar than the first amphipat, the latter has advantageously a higher than the otherwise recommended number of hydrophobic anchors per headgroup and vice versa. Molecular heterogeneity of the employed sugar amphipats again desirably increases the preferred number of hydrophobic anchors per sugar segment.

Further embodiments relate to the use of the described additives for modulating aggregate compositions comprising amphipats with n hydrophobic segments with a total of nC C-atoms each attached directly or indirectly, e.g. via glycerol backbone, to a phospho-, sulpho-, or arseno-headgroup, which is optionally derivatised, e.g. alkylated (as in fatty glycero-phospho-methyl-ester), coupled to an alcohol (as in fatty glycero-phospho-glycerol, i.e. phosphatidylglycerol), an amino-alcohol (as in phosphatidyl-ethanolamine or, more preferably, phosphatidyl-(N,N)dimethyl-ethanolamine), to an amino acid (as in phosphatidylserine), which can be further derivatised, etc. If the headgroup is zwitterionic, the positive charge, which normally resides on a ternary or quaternary amine (as in choline) is attached to the negatively charged part of the headgroup via a linker that preferably has between 2 and 6 C-atoms. Such first amphipat in the embodiments is often supplemented with another, similar or dissimilar amphipat, which is more polar if n=2 and less polar if n=1. If the first amphipat carries a glycerophosphocholine headgroup, the rule specified herein for selecting the second amphipat of fatty-polyoxyethylene type still applies if one treats, in the first approximation, such phospholipid as if it had 4EO per headgroup, i.e. to have n_{eff}˜4.

### 5.3.3 Additional Considerations

Many commercial amphipats/surfactants eligible for making preparations of the invention are a mix of (closely) related molecules with certain headgroup- or chain-length distribution, or both. The measured Ac and HLB values of such compounds reflect the distribution(s), in contrast to the simply calculated Ac value and HLB number, which presume monodispersity. Molecular polydispersity decreases the calculated effective Ac value, but leaves polarity units and HLB numbers unchanged. This is yet another reason for preselecting an aggregate component based on Ac value rather than on polarity units or HLB numbers according to the invention.

Some commercial amphipat products are a mix of BL- as well as ML-class molecules; it may then be unnecessary to add another ML-class molecule (such as a surfactant) to the preparations made from such compounds. Some nominally non-ionic products contain ionic contaminants; it may then be possible to benefit from (preferred) ion addition, even without extra charged amphipats inclusion into the preparation: drug-aggregate interactions and partitioning effects then need to be considered, however. This may yield a first to second amphipat molar ratio in the range of about 50:1 to about 1:25, more often in the range from about 20:1 to about 1:10, preferably in the range from about 10:1 and about 1:5 and more preferably in the range from 5:1 to 1:2.5. However, practically useful molar ratios outside these ranges are possible, especially if the bilayer-building and bilayer-destabilising amphipats are relatively similar. The preferred molar ratio typically decreases, i.e. more of the second amphipat is needed, if the first amphipat Ac and/or HLB number is closer to the lower BL-class criterion limit, and vice versa.

Some embodiments described herein contain no first amphipat that forms bilayer vesicles upon dispersion in an aqueous suspension and a lamellar phase upon water concentration reduction. Some embodiments contain no phospholipids at all. Most embodiments contain no cholesterol. Other embodiments contain no ethanol and/or no propylene glycol and/or are devoid of acetic acid, 2,2-dichloroacetic acid, acylated amino acid, adipic acid, alginic acid, ascorbic acid, L-aspartic acid, benzenesulphonic acid, benzoic acid, 4-acetamidobenzoic acid, boric acid, (+)-camphoric acid, camphorsulphonic acid, camphor-10-sulphonic acid, capric acid, caproic acid, caprylic acid, cinnamic acid, citric acid, cyclamic acid, cyclohexanesulfamic acid, dodecylsulphuric acid, ethane-1,2-disulphonic acid, ethanesulphonic acid, 2-hydroxy-ethanesulphonic acid, formic acid, fumaric acid, galactaric acid, gentisic acid, glucoheptonic acid, D-gluconic acid, D-glucuronic acid, L-glutamic acid, a-oxoglutaric acid, glycolic acid, hippuric acid, hydrobromic acid, hydrochloric acid, hydroiodic acid, lactic acid, lactobionic acid, lauric acid, maleic acid, malic acid, malonic acid, mandelic acid, methanesulphonic acid, naphthalene-2-sulphonic acid, naphthalene-1,5-disulphonic acid, 1-hydroxy-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, tartaric acid, thiocyanic acid, p-toluenesulphonic acid, undecylenic acid, and valeric acid. Additionally, some embodiments contain no magnesium-, calcium-, potassium-, zinc- or sodium-hydroxide and also no arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methylglucamine, hydrabamine, imidazole, lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2(hydroxymethyl)-1,3-propanediol, and tromethamine (TRIS).

Unlike known aggregate formulations, each embodiment of the invention includes at least one aggregate-improving, relatively hydrophilic, additive. For the charged aggregates of the invention, the additive can be chosen to be a suitable dissociated salt that may also act as a buffer, partially dissociated microbicide, antioxidant, fragrance, etc. In many embodiments, such dissociated salt is a preferred ion. If the ion is an at least partially ionised acid, then the acid can be a linear or a branched fatty acid that advantageously carries, e.g., around 2 to around 7 C-atoms per carboxylic residue. Each extra polarity-increasing side-chain, or atom, within the molecule allows total C-atoms number per molecule to be increased; the increase may amount to e.g. around 1.5-2.5 more C-atoms per carboxylic residue. The introduction of apolar, e.g. halide atoms, conversely lowers the preferred number of C-atoms per carboxylic residue per molecule.

A non-limiting list of acids useful for the invention includes acetate, isobutyrate and isovalerate, isocaproate, diethylacetate, ethylvalerate, methyl hexanoate, hydroxyisovalerate, leucate, succinate, glutarate, adipate, pimelate, suberate, azelate (nonanedioate), methylmalonate (iso-succinate), ethylmalonate, propylmalonate, methylsuccinate, ethylsuccinate, propylsuccinate, pyrotartrate, methylglutarate, ethylglutarate and propylglutarate, methyladipate, and ethyladipate, methylpimelate, methylsuberate, methylazelate, dimethylmalonate, diethylmalonate, dipropylmalonate, dibutylmalonate, dimethylsuccinate, diethylsuccinate and dipropylsuccinate, dimethylglutarate, diethylglutarate, the practically acceptable dimethyl- and diethylhexanedioates, dimethylheptanedioates, but also oxoisocaproate, methyl-2-hydroxyisocaproate, citramalate, etc., with consideration of the various possible side-chain(s) attachment(s) yielding an acceptable distribution ratio (e.g. considering alpha- and beta-forms of isovalerate, 2,2- and 2,3-dimethylbutanedioate, 2,2-, 3,3-, or 2,4-dipropylpentanedioate, etc.) and making the best use of stereochemistry effects on the considered acid dissociation constant.

Furthermore, some embodiments contain phosphoric acid esterified with at least partially hydrophobic side chains, as in methyl-, ethyl-, propyl-, butyl-, pentyl-, hexyl-, heptyl-, octyl-, nonyl-, decyl-, or in dimethyl-, diethyl-, dipropyl, dibutyl-, pentyl-methyl-, pentyl-ethyl-, penthyl-propyl-, hexyl-methyl-, hexyl-ethyl-, heptyl-methyl-ester derivatives, preferably choosing total C-atoms number in such molecules to be between 4 and 10. Derivatives having branched side chains or side chains with an oxo- and especially (terminal) hydroxy-group represent valuable components of some preparations of the invention as well. Similar derivatisations are advantageously used in other embodiments where the starting phosphoric acid is replaced by phosphorous, sulphuric, or sulphonic acid.

Embodiments containing negatively charged aggregate compositions further include a base, e.g., hydroxyethyl)amino]-2-(hydroxymethyl)propane-1,3-diolaminoglycol, TRIS, 2-amino-2-ethylpropane-1,3-diol, 2-(2-aminoethyl)-2-(hydroxymethyl)propane-1,3-diol, 2-amino-3-2-aminoisobutanol, methoxy-2-(methoxymethyl)propan-1-ol-2-amino-3-methoxy-2-methylpropan-1-ol-2-aminoisobutanol, 4-amino-2-tert-butyl-2-methylbutane-1,3-diol, trolamine, diethanolamine, diethylenediamine, ethylenediamine, ethylamine, diethylamine, ethanolamine, methyl-ethanolamine, methyl-diethanolamine, ethyldiethanolamine, dimethylethanolamine, ethylethanolamine, diethylethanolamine, propanolamine, N,N-bis(2-hydroxypropyl)butanamine, pyrrolidine, epolamine, 1-pyrrolidin-1-ylethanol, 1-(pyrrolidin-1-yl)propan-2-ol, pyrroline, pyrrole, imidazole, methylmidazole, dimethylimidazole, trimethylimidazole, piperidine, piridine, piperazine, methylpiperazine, and ethylpiperazine, piridazine, pyrimidine, pyrazine, phenylamine, and cyclohexylamine, but the normally zwitterionic glycine, bicine and tricine, aspartic acid and Pipes are also found in certain formulations according to the invention.

The number of C-atoms per charged residue, e.g. an amino-residue, on many particularly useful bases is between at least 2 and around 7-8. Introduction of additional polar (e.g. oxo- or hydroxy-) group(s) increases the preferred C-atoms number by up to around 1.5-2.5 per molecule and charged residue. Introduction of one or more apolar residues, such as halide atoms, lowers the preferred tolerable number of C-atoms per charged residue and molecule as discussed elsewhere herein. In some embodiments, the concentration of the at least one aggregate-improving additive increases with the aggregates dry mass in a preparation and vice versa. Moreover, the higher the chosen dry mass, the less hydrophilic may be the chosen additive, to ensure a sufficiently proper distributed additive/amphipat molar ratio.

### 5.3.4 Applications

Some embodiments relate to at least one aggregate-associated (i.e. aggregate-adsorbed, -bound or -encapsulated) agent for treating at least one skin condition. Examples are acne, dermatitis (e.g. seborrhoeic dermatitis (including dandruff, otitis externa), discoid eczema, pompholyx, etc.), atopic dermatitis (e.g. atopic and childhood eczema), papulosquamous disorders (such as lichen planus, granuloma annulare, cutaneous sarcoidosis), disorders of skin colour (e.g. melasma or vitiligo), urticaria, angio-oedema and other inflammatory skin disorders, erythema multiforme, Stevens-Johnson syndrome and toxic epidermal necrolysis, nail disorders, rosacea, hidradenitis suppurativa and other disorders of the apocrine sweat glands or sweating disorders, disorders involving the skin's blood and lymphatic vessels (such as dilated blood vessels, cutaneous blood vessels inflammation, oedema and lymphoedema, blushing and flushing reactions, cutaneous striae, panniculitis, lupus vulgaris and lupus erythematosus, scleroderma, morphoea and related conditions, lichen sclerosus and related conditions, dermatomyositis, immunobullous (blistering) disorders of the skin (such as bullous pemphigoid, pemphigus, dermatitis herpetiformis), pruritus and skin itching, epidermolysis bullosa, malignant or benign infiltrations of the skin (such as tumours and cysts of the skin and appendages, but also keratolytic agents, antihistamines, and the like).

Certain embodiments contain other pharmacological agents, such as anti-infectives, including antibiotics and antivirals. Popular representatives of the former class are aminoglycosides, beta-lactames, including penicillines (such as amoxicillin, clavulanic acid, tazobactam, flucloxacillin, piperacillin, and less preferably benzylpenicillin, phenoxymethylpenicillin), cephalosporines (such as ceftobiprol, cefepim, cefixim, cefoperazon, cefotaxim, cefpodoxim, cefprozil, ceftazidim, ceftriaxon, or ceftibuten), carbapenemes (i.e. thienamycine, doripenem, ertapenem, imipenem, meropenem), monobactames (such as aztreonam), chinolone (such as trovafloxacin, levofloxacin, moxifloxacin, ofloxacin and ciprofloxacin), chloramphenicol, folic acid antagonists (such as sulfonamides and methotrexate), fusidic acid, glycopeptide-antibiotics (such as vancomycin and teicoplanin), lincosamides (such as clindamycin and lincomycin), macrolides (such as azithromycin, clarithromycin, erythromycin, roxithromycin, spiramycin), nitrofuranes (such as nitrofurantoin, nitrofural and nitroimidazole), oxazolidinones (such as linezolide), phosphonic acid derivatives (such as fosfomycin=phosphonomycin), polymyxines (such as colistimethate and polymyxin B), polypeptide-antibiotics (such as bacitracin, gramicidin, tyrothricin), tetracyklines (such as demeclocyclin, doxycyclin, lymecyclin, minocyclin, minocin, tetracyclin and glycylcycline tigecyclin), however, numerous other drugs are suitable for incorporation into the presently described aggregate compositions.

Useful antiviral agents in the preparations of the invention include, but are not limited to nucleoside-analogues (such as aciclovir, brivudin, cidofovir, famciclovir, foscarnet, ganciclovir, idoxuridin, penciclovir, valaciclovir, valganciclovir, tromantadin), neuraminidase antagonists (such as amantadin), anti-HIV drugs (including CCR5-antagonists, antifusogenic drugs, as well as integrase, HIV proteease and reverse-transcriptase inhibitors) adefovir, ribavirine, in addition to less prevalent drugs for viral infection treatment. Glucocorticosteroids suitable for use in the aggregate compositions include e.g. betamethasone, cloprednole, cortisone, cortivazole, dexa-methasone, deflazacort, fluocortolone, hydrocortisone, meprednisone, methyl-prednisolone, paramethasone, prednylidene, prednisolone, prednisone, rimexolone, triamcinolone acetonide, however, most steroids may be readily incorporated into the described adjustable aggregates according to the present teachings.

Particularly attractive (local) anesthetics useful for the embodiments of the invention include, but are not limited to articaine, benzocaine, bupivacaine, butacaine, butoxycaine, acyl-caine (e.b. methyl, ethyl, propyl, butylcaine and iso-butylcaine, pentyl- and iso-pentyl as well as hexyl-caine), chloroprocaine, cornecaine, cyclocaine, dimethocaine, dyclocaine, etidocaine, fomocaine, hydroxyprocaine, leucinocaine, lidocaine, mepivacaine, oxethazaine, oxybuprocaine, oxypentacaine, parethoxycaine, piperocaine, piridocaine, pramocaine, prilocaine, procainamide, proparacaine, propiocaine, propoxycaine, pyrrocaine, quinicocaine, ropivacaine, tetracaine, and tolycaine.

Another embodiment aims at ensuring high drug payload and/or increasing drug stability in the preparation. In other embodiments, amphipats with ether- or amide-bond(s) between fatty chain(s) and headgroup are employed to allow lower pH usage for either of the purposes by increasing the tolerance of the aggregate composition to acidic and alkaline surroundings where ester-bonds can be cleaved fairly rapidly. This can moreover help achieve a higher payload of the aggregates of the invention, either directly or indirectly. For example, known deformable phosphatidylcholine-polysorbate vesicular aggregates have a relatively small, solubility-limited, payload at pH≧5 (4.5), wherein the carriers have been described as being reasonably stable. However, such vesicles have relatively short, amphipat stability limited, shelf-life at more acidic pH values, e.g. pH≦5 (4.5), wherein the drug payload, e.g. terbinafine, solubility is higher. In contrast, terbinafine formulations made according to the invention from non-ester amphipats, e.g. from the polyoxyethylene- or polyglycerol fatty ethers with an average area per chain of between around 0.43 nm² and around 0.5 nm², solve both problems, at least by providing an improved shielding of the ester-bonds on the long-headed amphipats compared to that achieved using phosphatidylcholine when the preparation pH is not too acidic.

The target pH selection of the disclosed aggregate compositions depends primarily on the desired ionisation state of the main formulation components and on the resulting preparation stability. For example, ester bonds of zwitterionic phospholipids degrade exponentially more rapidly with an increasing preparation-pH difference from pH=6.5. “Sheltering” such bonds in a bilayer does not always suffice to diminish this problem to an acceptable degradation rate level. Replacement of the ester-bonded molecules with the corresponding amide- or ether-derivatives can offer a better solution. The pH choice is sometimes dictated by solubility, especially of the pharmaceutically active ingredients present in an aggregate composition. Unless specified otherwise, the aggregate pH is typically between about pH=2.5 and about pH=9.5, between about pH=3 and about pH=8.5, or between about pH=4 and about pH=7.5. More specifically, suspensions of neutral aggregates should preferably be kept around pH=6.5. Cationic formulations should preferably be more acidic (i.e. pH 6.5) and anionic formulations more alkaline (pH 6.5), the desired difference increasing with increasing charge surface density on aggregates of the invention.

Preparations comprising one or several ionisable ingredients, which contribute to the desired system properties only when charged, should ideally have a pH sufficiently different from pK of such formulation key compounds. This ensures that at least 50% and more preferably 75% of all dissociable ingredients in the preparation, or at least at the site of application, are ionised. In practice, a preferred pH should be in the range between around pH≦(pK_{a,mem}+0.5) and around pH≦(pK_{a,mem}−1.5) for positively charged aggregates and around pH≧(pK_{a,mem}−0.5) and around pH≧(pK_{a,mem}+1.5) for negatively charged aggregates. pKₐ (and in case pK_{a,mem}˜PK_{a,ass}) of ionisable additives of the invention plays a role as well. Ideally, the preferred pH range criteria specified above for aggregates should also be met for such key ionisable additives. Otherwise, general knowledge and this disclosure must lead to a compromise. Choosing an acidic preparation for administration on the skin surface (with pH˜5±1) should also be taken into consideration.

Another embodiment relates to compositions that include at least one co-solvent, which is frequently at least a partially polar (organic) fluid (such as an alcohol, e.g. ethanol, propanol, ethylene glycol, glycerol, etc). In contrast, other embodiments contain no alcohol and may even be entirely co-solvent free. Some, but not all, embodiments contain at least one antioxidant (such as metabisulphite, di-tert-butyl-phenol, BHA, BHT, etc). In some embodiments, at least one fragrance (such as linalool, myrcene or mircenol, 1-hexanol, menthol, etc.) is included, but it is also possible to design and use embodiments without a fragrance. Some embodiments contain no other aggregate builder with at least 10-fold different solubility in the suspending medium than the main bilayer forming amphipat. Some embodiments are prepared from amphipats having relatively short hydrophobic chains with around 12 C-atoms each, which may be especially beneficial when a drug to be delivered by the aggregate composition is relatively lipophilic. Other embodiments are prepared from the amphipats having at least one unsaturated hydrocarbon chain, which can be linear or cyclic, simple or derivatised, and in the former case typically contains around 18 C-atoms joined with a single and up to three, but preferably only two and ideally just one, double bond(s) per aliphatic chain; molecules with several hydrophobic chains should carry at least one unsaturated, or otherwise fluidised, aliphatic chain. Additional embodiments involve amphipats with relatively long polar heads, some of which consequently form relatively inflexible bilayers on their own. Embodiments in which such amphipats are combined with at least one bilayer softening ingredient lend themselves particularly well for delivering or controlling release of amphiphilic agents and/or moderate lipophilic agents, which then partition preferentially into the resulting interfacial region. Some embodiments of the invention are made mainly to facilitate aggregate manufacturing and/or suspension sterilisation via filtration through a suitable filter membrane. Some embodiments of the invention are made so as to ensure long-term aggregate stability supported by bilayer flexibility (which can be controlled, and if desirable increased, by additives of the invention).

The invention recognizes that designing aggregate compositions having a significant proportion of ionic amphipats and/or drugs requires special skill for ensuring a proper balance between electrostatic effects enhancement (achievable via ionic strength lowering) and increased ions adsorption to said aggregates (achievable via (preferred) ions concentration increase). In parallel, the chosen pH should maintain a reasonably constant net charge on the ionised components of the preparation. For example, some aggregate formulations have ions with a total ionic strength up to around 0.3 mol L⁻¹. More frequently, ionic strength is up to around 0.15 mol L⁻¹, preferably up to around 0.075 mol L⁻¹, and even more preferably up to around 0.05 mol L⁻¹. Ionic strengths of around 15-25 mmol L⁻¹ can be useful to allow for any ionic strength increase that may occur during partial drying at a non-occluded application site, but then more often than not some other excipients (e.g. a polymeric thickener) contribute to the overall buffering capacity of the preparation. (Essentially immobile ions, e.g. charges on polymeric viscosity builders or on the aggregates of the invention, are excluded from ionic strength calculation for purposes of the invention.) A non-exclusive list of ions that qualify as preferred ions for use in the invention is provided elsewhere in the text.

In another embodiment, (preferred) ions are introduced into a preparation of the invention as a salt of an ionisable component of the formulation, e.g., as a suitable salt of the employed drug and/or ionic amphipat. More specifically, it can be advantageous, first, to form the preferred ion salt of a suitable formulation component and, second, to introduce such salt into the preparation, instead of beginning with an uncharged drug/amphipat form and then ionizing this form with a suitable buffer (acid or base) addition. Selecting the first option is particularly important where the concentration of charged drugs/amphipats in the final aggregate preparation is proportionately high, i.e. when such components contribute ≧05 mol L⁻¹ to the overall ion-strength of the preparation. If no desirable salt form is readily available, one can prepare such salt from free-base or -acid using no more than general knowledge and published advice (see, e.g., Handbook of Pharmaceutical Salts. Properties and Use, 2^{nd} ed. (Stahl and Wermuth, eds., 2008, VHCA, Zurich, and Wiley-VCH, Weinheim). In some embodiments, (preferred) ions are introduced at a higher than final concentration during the starting suspension preparation step, and then brought to the final ionic strength by adding a less concentrated solution, or even ion-free solvent, or water, during subsequent aggregate manufacturing.

Preparations with a significant proportion of ionic amphipats may additionally benefit from inclusion of strongly repulsive, bilayer-compatible nonionic amphipats (relative molar ionic/nonionic amphipats ratio from about 1:2 to about 1:50, preferably from about 1:3 to about 1:20, even more preferred from about 1:5 to about 1:9), to improve the resulting aggregate stability during storage and/or at the site of application onto a surface. The aggregate-stabilising nonionic amphipats can be present in the aggregates of the invention at relative weight concentration from about 0.01 wt.-% to about 25 wt.-% of the total amphipat weight, often from about 0.05 wt.-% to about 10 wt.-% and preferably from about 0.1 wt.-% to about 5 wt.-%.

Many embodiments herein disclose submicroscopic aggregates, i.e. vesicles smaller than 0.75 μm and typically smaller than 0.4 μm, but the principles disclosed by the invention apply to larger aggregates as well. The average aggregate diameter in preparations of the invention is normally below 0.6 μm, more often below 0.3 μm and preferably up to around 0.175 μm. The lower size limit is between around 20 nm and around 50 nm. These preferred values typically correspond to suspension turbidity (i.e. to the measured optical density corrected for any absorption) up to around 1.8, more preferably up to around 1.2, and most preferably up to around 0.8±0.4 (1 cm light-path, 800 nm incident light wavelength). The described vesicular aggregates have typically fewer than 10 bilayers per aggregate on the average, more often not more than 5, preferably merely up to 3 and even more preferably only 1-2 bilayers, on average.

Any formulation of the invention may optionally contain antimicrobials/antifungals, other preservatives, antioxidants, chelators, co-solvents, emollients/humectants, enzyme inhibitors, fragrances and even flavours, as well as thickeners, either alone or in any suitable and practically, e.g. pharmaceutically, acceptable combination.

Amphipats having fluid chains at normal skin temperature (i.e. around 30±2° C.), and ideally under all reasonably expected storage conditions, are preferred in the preparations of the invention made for epicutaneous application. Other applications depending on the sufficient adaptability of the aggregates also rely on chain fluidity, which may only be required at the application temperature; the latter must therefore be specified, and if necessary re-defined, appropriately. This observation explains why most embodiments reported herein describe amphipats having fluid lipid chains at least above 20° C. To reach this goal, preparations of the invention preferably contain a significant proportion of intermediate chain-length amphipats (C8 to at most C14) or employ amphipats with long but unsaturated, branched or derivatised chains, such as C14:k, C16:k, C18:k, C20:k, or even C22:k, where k=1, 2 (or 3). C14:1, C16:1, C18:1 or C18:2 chains, or iso-C14, iso-C16, or iso-C18 are more preferable in this respect than are C18:3 or C20:3, C20:4 chains, or alkenoxy chains, etc. It is therefore more beneficial to combine saturated and unsaturated chains of similar lengths, attached to physically similar headgroups (volume-, charge-wise, etc.) then to blend molecules with appreciably different tail lengths (nC−difference≧4), which can prompt molecular separation in a preparation.

When using the invention just to accelerate and/or ease vesicularisation, ordered chains in the final bilayer state may be acceptable. (Ordered chains may be desirable when the aggregate-associated payload resides predominantly in the vesicle interior, from which transbilayer leakage should be minimized.) Since fluid-chain bilayers form vesicles easier and faster than ordered-chain bilayers, it is preferable to prepare suspensions at a temperature above the chain-melting temperature of the mixed amphipat bilayer (obtainable or calculated from publicly available information). Choosing a suitable manufacturing temperature must also consider the temperature sensitivity of polar headgroups conformation and, in particular, hydration. (Hydration of nonionic headgroups more often than not decreases upon heating, e.g., often more so than for the relatively less hydrophilic headgroups (polyoxyethylene or polyoxypropylene>>polyglycerol˜(poly)sugar). In contrast, zwitterionic and especially ionic headgroups have typically positive temperature gradient of hydration.) Choosing manufacturing conditions so as to reach the desirable Ac as specified herein may be commendable as well.

In another embodiment, amphipats with positive- and amphipats with negative-temperature dependency of solvation are combined to minimise the resulting mixture temperature sensitivity. A desirable working temperature is between 4° C. and 95° C. during manufacturing, and subsequently between room temperature and 65° C., and most often between around 30° C. and around 37° C., except for storing (ideally at a low temperature, e.g. 4° C., and for practical reasons most often at room temperature). The manufacturing temperature in some embodiments is optionally lowered before and/or during suspension homogenisation, especially if the employed amphipats hydration decreases with temperature, as is the case with polyoxyethylene- or polypropylene-esters, -ethers, or -amides; the manufacturing temperature is optionally increased before and/or during homogenisation of the suspended amphipats with a positive temperature effect on the amphipat headgroup hydration, e.g. phosphatidylcholines, phosphatidylglycerols, dimethylphosphatidyl-ethanolamine or phosphatidic acid and sphingolipids, to expedite vesicularisation or aggregate loading with drugs.

Producing certain preparations of the invention entails at least one temperature change during the manufacturing process, either to ensure faster dissolving of the formulation components and/or to modulate the effective area per hydrophobic chain of the aggregate-forming amphipats, and thus to either accelerate vesicularisation and/or to lower the energy input required for reaching the targeted final aggregate size and/or to achieve a more favourable final aggregate size or molecular distribution within the preparation.

Manufacturing the adaptable aggregates according to the invention can furthermore include a change of the solution or suspension pH either once or several times to manipulate, in particular, the ionisation of the aggregate-associated components, e.g., to thereby ensure either faster dissolving of the formulation components and/or to modulate the effective area per hydrophobic chain of the aggregates-forming amphipats. Both accelerate the vesicularisation process and/or lower the energy input needed to reach the final aggregate size and/or to ensure a more better final aggregate size or molecular distribution in the preparation of the invention compared to the aggregate manufacturing processes not involving such changes.

In certain embodiments, the preparations are produced in sequential steps, e.g., first, by separately combining the hydrophilic and the hydrophobic formulation ingredients in two separate, reasonably homogeneous, and preferably fluid, mixtures; second, by controllably combining the two mixtures. This is preferably done by drawing-in or injecting, or potentially by dripping-in or distributing the less voluminous preparation or solution over the surface of the bulkier solution, which typically contains the suspending medium. The rate of the stirring, drawing-in or injecting, and/or the optional additional, externally generated, homogenisation stress (used to make an acceptably uniform suspension from two immiscible solutions and to gain acceptably small aggregates in the final product) is thereby adjusted and controlled so as to achieve the desirable average aggregate size and distribution in the final aggregates suspension, which is then optionally thickened by adding a suitable viscosity modifier.

All lists and ranges herein merely exemplify and do not limit the selection of a particular feature, whether such lists and ranges pertain to amphipats, anions, cations, and concentrations thereof, combination choices, ratios, pH values, temperatures, and the like. Any skilled practitioner will understand, e.g., how to extend the provided exemplary list of suitable additives by determining suitable charged or uncharged additives for a particular application based on the rules and other selection criteria provided herein.

### 5.4 Aggregate Preparations

Depending on their composition, prevailing physical properties and presentation form (pure substance or its solution/suspension), the amphipats and additives useful for making the disclosed preparations can be solid, waxy, or fluid. To ensure adequate mixing of all formulation components, they should be liquid/liquified before combination. Such liquefaction is normally carried out separately for the lipophilic/amphipathic ingredients and for the water-soluble ingredients of the preparations.

### 5.4.1 Functional Testing

Methods suitable for characterizing an embodiment of the invention in an ex vivo system are published (cf. in Wachter et al., 2008 (op. cit.); Cevc et al., 2008, Int J Pharm 360: 18-28; Elsayed et al., op. cit.). Special considerations for making the aggregate compositions according to the invention are described below.

Effects of soluble additives, such as ions, on aggregate adaptability can be confirmed with different experimental methods. One can monitor, e.g., enforced small-aggregates formation kinetics (which provides information on vesicularisation time) and the resulting vesicle stability. To ensure essentially constant bilayer surface characteristics, in particular a constant surface charge density, component concentration must be considered properly. Monitoring the test suspension turbidity as a function of time during sonication with a constant power volume density and at constant pH, temperature, and amphipat concentration can meet this objective.

A first consideration is that the chosen pH is far enough from the tested aggregate surface pK_{a,mem} to exclude ionisation changes during a test; otherwise, experiments must be conducted at two or more constant, yet different, pH values and then analysed together allowing for the pH effects. A second consideration is to conduct all experiments with a similar preparation volume using similar power setting (nominal power; duty cycle) and transducer diameter, to ensure a constant input energy volume density. If different suspension volumes are studied, the power-input or vesicularisation time requires appropriate normalisation to compensate for the difference. For simplicity, the key results of such measurements are expressed herein in terms of vesicularisation time. (Note that an unusual viscosity increase can reveal presence of long structures (e.g. thread-micelles) alongside bilayer vesicles.)

In a simpler and more qualitative alternative assay, one compares the test sample turbidity with an appropriate opalescent/transparent reference suspension turbidity. A series of readings taken for each representative sample at different times can likewise reveal temporal stability. Experimental data generated with this method confirmed the independency of the conclusions drawn from individual turbidity readouts for the preparations of the invention: within experimental error limits, the difference between the time required to generate more or less, but always similarly turbid suspension is generally the same. Vesicularisation time sensitivity to the tested suspension volume, or to the used energy input density, plays no role since the relative difference analysis suffices for identification and characterisation of the inventive preparations.

The vesicle adaptability (bilayer deformability) in representative suspensions expressed in terms of vesicularisation times, deemed to meet the sufficient adaptability requirements of the invention, was compared with the results of the experiments in which the enforced tested suspensions flux through a semipermeable, porous (polycarbonate or metallic) filter was measured, the pores being ≦50% smaller than the average aggregate diameter (typical pore diameter: 20-50 nm, depending on the experiment). The filter-penetrability, calculated by dividing the flux with the filter area and the flux driving pressure, correlated nearly perfectly with the corresponding vesicularisation time. A graphical illustration of the correlation is presented in Cevc, 2012, J. Contr. Rel. (op. cit).

Aggregate size stability can be tested optically. The simplest method is to compare the test sample turbidity with a reference sample turbidity known to be stable. If desired, the test sample turbidity can moreover be quantified at a fixed incident light wavelength (typically selected between 400 nm and 800 nm, most often at 800 nm) for quantitative control. For even greater accuracy, the turbidity spectra of the dust-free preparations prefiltered through a 0.2 μm pore filter are recorded spectrophotometrically outside the range of incident light absorption (cf. Elsayed & Cevc, 2011, op. cit.), or corrected for such absorption contributions to the spectrum.

### 6. EXAMPLES

### 6.1 Poorly Deformable Neutral Vesicles (“Liposomes”) as a Negative Control

Double-Chain Uncharged (Zwitterionic) Amphipats in a Suspension.

The first type of reference suspension, acting as a negative control, contained quasi single-component vesicles made from a common glycerophospholipid, phosphatidylcholine (herein >95% pure soybean-extract with Ac˜0.35 nm²). Such bilayer vesicles (liposomes) have a low adaptability and therefore cannot cross pores significantly (here >50%) smaller than their own diameter; this fact is well-known, as are the preparation methods for making unilamellar vesicles from such lipid, such as the sonication of a crude dispersion of the lipid in a suspension medium.

Table 1 reports the vesicularisation time (t_{vesicle} or tᵥₑₛ) needed to obtain an opalescent, 10 w-% suspension of soybean phosphatidylcholine vesicles (diameter around 100 nm) from an originally crude suspension in an inorganic buffer with 100 mM ionic strength as a function of the bulk suspension pH. Such time is constant (t_{vesicle}˜1250 s) within experimental error limits (around ±200 s) at least in the range 4≦pH≦8.5, where ≦90% pure soybean phosphatidylcholine is uncharged. The somewhat faster small vesicle formation in alkaline suspensions (pH≧9) is explained by a slight ionisation of the bilayer-bound fatty acids present in the preparation in initially, or else generated by phosphatidylcholine hydrolysis during sonication at such high pH. The related vesicularisation-time shortening at pH≦3, which is also reflected in the practically decisive relative vesicularisation time, tᵣₑₗ, can be explained by the phosphate groups protonation at pH near pK_{a,mem}≦1-3, which creates an excess of net positive charges from the choline groups on phosphatidylcholine headgroups. At temperatures up to around 60-70° C., vesicularisation time is not particularly temperature sensitive. In contrast, temperatures >around 75° C., and especially >around 90-95° C. appreciably shorten the vesicularisation time of phosphatidylcholine.

In neutral and moderately acidic pH ranges, the choice of the tested suspension buffer thus apparently plays no practically meaningful role in small phosphatidylcholine-liposomes formation. In contrast, the employed organic buffers can expedite formation, and thus arguably adaptability and deformability, of partially charged lipid vesicles in alkaline suspensions, albeit to a different extent. Uncharged phospholipid (di)esters are most stable around pH˜6.5, and degrade exponentially faster at pH above or below such optimum. It may consequently be practically useful to exploit the observed additive effect at the higher or lower pH during suspension preparation to expedite small aggregates formation in a transiently acidified or alkalinated suspension.

Single Chain Uncharged (Nonionic) Amphipats in a Suspension.

The second kind of control, or reference, suspensions were prepared from nominally single component aggregates comprised of nonionic surfactants from the BL-class (defined herein), or alternatively, from BL- and ML-class amphipat mixtures with a similar calculated average area per chain, Ac. (Single-chain molecules with Ac near the lower end of the range specified for the BL-class amphipats herein either form a lamellar phase too stiff to allow proper dispersion or phase-separate. In contrast, phosphatidylcholine and many other double-chain amphipats with such an Ac can be finely dispersed but require significant energy input to the end.)

Detailed compositions of the representative suspensions tested are specified in Table 2, which also reports the corresponding vesicularisation times (t_{vesicle}) and Ac values. Like phosphatidylcholine dispersions, suspensions made from amphipat (mixtures) with Ac in the lower or middle part of the BL-class range require long sonication times to become transparent, if at all (for preparations with 10 wt.-% total amphipat concentration: t_{vesicle}>300 s). Moreover, suspensions containing amphipats with a low calculated average Ac do not form small vesicles; such suspensions thus remained opaque even after long sonication. On the average, it is easier and faster to obtain vesicles from the amphipats with more than one chain per molecule than from the single-chain amphipats that have a comparable area per chain. (NB: molecules that offer multiple options for hydrophobic chain and/or polar groups coupling are often polydisperse. This aggravates, and may preclude, direct comparisons between the related aggregate products originating from different sources (cf. Exs. 28, 29, 44).

### 6.2 Adaptable, Mixed Amphipat, Neutral Vesicles as a Positive Control

One useful positive control reference includes suitable (i.e. surfactant rich but not solubilised) blends of zwitterionic phosphatidylcholine, a BL-type amphipat, and at least one non-ionic ML-type (i.e. surfactant-like) amphipat with (a) long acyl or alkyl chain(s). Another useful positive control include suitable non-ionic amphipat blends with the average Ac value below, but close to, the specified upper BL-class Ac limit. Measurements using any such suspended mixture consistently reveal that the time needed to achieve the suspension transparency—and thus to transform the originally large aggregates into small and highly adaptable vesicles—decreases with increasing average area per surfactant chain, Ac (compare Tables 2 and 3). In fact, when the average Ac is near the upper BL-class limit, which normally increases with amphipat polydispersity, the shortest achievable vesicularisation time under the chosen experimental boundary conditions is around 30±20 s. This is one of the hallmarks of ultradeformable vesicles in a 10 wt.-% uncharged amphipat suspension, since the lowest achievable t_{vesicle} value generally signals bilayer adaptability close to its thermodynamically limited maximum. As shown in Tables 2 and 3, this range is comparable to the corresponding phospholipid/nonionic surfactant ratios published previously for similar aggregate compositions tested in a penetration assay (Wachter et al., 2008, op. cit.). The conclusion holds true for various chain lengths a so long as the bilayer interior is fluid and the resulting greater/lower degree of shorter/longer chains disorder is allowed for. (It is noteworthy that the studied reference suspension behaviour typically reflects the known phase boundaries of the involved amphipatwater mixtures, e.g. the proximity of a bicontinuous cubic phase points toward fast vesicularisation/high adaptability of the studied aggregates and proximity of an inverse phase suggests long vesicularisation time, and thus aggregate stiffness.)

6.3 Aggregates Containing Negative Amphiphiles (e.g. Lipophilic NSAIDs)

Known reports disclose increased adaptability of mixed vesicles upon certain charged drugs binding to aggregate surfaces. The synergy between adaptability-increasing effect of the charged drugs and surfactants is known in the art too, and best explored for NSAIDs. Some embodiments of this invention therefore include NSAIDs, and then use published formulations for comparison. Consistent with these reports, most of the tested suspensions contained 10 wt.-% of total amphipat (BL+ML, including the drug), unless specified otherwise. (However, no membrane destabiliser other than the drug itself was included into the initial test formulations.) If the included drug was an NSAID, its contribution to the combined amphipats mass was initially set at 30 rel. wt.-%. (The basic characterisation relied on a sonication assay, complemented by the narrow-pore barrier-penetration assay known in the art.)

Table 4 reports a set of representative data with a focus on the NSAID ketoprofen (molecular weight: MW=254.30; de/protonation constant in the bulk: pK=4.01; water-membrane partition ratio for the neutral and ionised drug forms: log Pₘₑₘ^{N}=3.28 and log Pₘₑₘ^{I}=1, respectively; de/protonation constant in a membrane: pK_{a,mem}=5.96, all pertaining to T=310 K and for phospholipid plus drug concentration of ˜10 w-%) suspended in various 100 mM buffers. The provided distribution ratios given in Table 4, and other tables herein, were calculated from the reliable experimentally published pK and pK_{a,mem} values and the averaged theoretical log P^{N} value, assuming log(P^{I}P^{N})=−1.25. These results confirm, first, that only the ionised drug acts as a bilayer deformability enhancer: the formulations with pH<<pK_{a,mem} prepared from ketoprofen/soybean phosphatidylcholine (3/7 weight/weight ratio; Ac(PC)˜0.35 nm²) are at least as rigid, if not stiffer and more difficult to disperse finely, than the corresponding drug free phosphatidylcholine vesicles (compare Tables 1, 2 and 4). A second conclusion is that the desirable drug-effect on bilayer adaptability/vesicularisation time plateaus when the pH appreciably exceeds pK_{a,mem} (i.e. for ketoprofen pH>6-7, dependent on the boundary conditions/buffer used), corresponding to between around 60 mM and around 110 mM charged ketoprofen (given the used total drug concentration of around 120 mM and pK_{a,mem}=6). A third conclusion is that the charged drug-partitioning—or better: distribution—ratio dominates over the buffer distribution ratio between the aqueous bulk and the studied aggregates. Despite this the buffer can significantly affect aggregate properties.

Replacing the phosphoric acid based buffer with a different inorganic buffer does not markedly improve the vesicularisation ability and adaptability of the resulting aggregate preparations. In fact, some inorganic buffers may deteriorate this situation. Exchanging phosphoric acid with sulphurous acid buffer, e.g., lowers the resulting drug-loaded vesicles adaptability by a factor around 2 (cf. Table 4). This is explained in terms of the distribution ratio that is slightly lower for phosphoric acid than for sulphurous acid. Results measured with various organic buffers in ketoprofen and soybean phosphatidylcholine suspensions conform with this observation (Table 4). These results also reveal that organic ionic additives (e.g. suitable buffer ions) can improve aggregate adaptability by at least a factor of around 2, even if the vesicles are rather adaptable to begin with.

Implications of the KT+SPC related data shown in Table 4 are obscured partially by the drug ionisation changes and by multiplicity or complexity of molecular charge distribution. From Tables 1 and 4 one can nonetheless deduce that some of the tested organic buffers, which carry at least some positive charges at the chosen pH (e.g. 6<pH<7.5), shorten vesicularisation time. Over a certain range, this effect is roughly proportional to the ionic molecule distribution ratio. The effect of organic additive on aggregate vesicularisation-time is clearer in the aqueous suspensions of ketoprofen fatty-polyglyceride (Emulsogen OG®, Clariant) and polysorbate 80 mixtures (10 wt.-% total amphipat; Emulsogen/polysorbate=1/1 wt./wt., Ac˜0.42 nm²; 30 rel. wt.-% ketoprofen in total). The corresponding data provided in Table 4 support the notion that increasing distribution ratio increases aggregate adaptability and colloidal stability, at least within certain limits.

For relatively hydrophilic additives, the beneficial effect of adaptability improving additives may be boosted by lowering the selected additive concentration (e.g., to 25 mM or 50 mM; cf. Table 6). This gain is enhanced if the aggregates leave more space for improvement, as in preparations with relatively low ketoprofen content (data not shown).

A bilayer softening effect of another NSAID, indomethacin (MW=291.43; log Pₘₑₘ^{N}˜3.8, log Pₘₑₘ^{I}˜1.5; pK=4.5; pK_{a,mem}˜6.6, best estimates) depends qualitatively similarly on ionic additive selection (cf. Table 4), thus further supporting the conclusions provided by this invention. Starting with a crude drug-phosphatidylcholine suspension, shorter vesicularisation times are measured with suspensions in a well chosen organic buffer (e.g. morpholinoethanol) than with suspensions in an inorganic (e.g. sulphite or phosphate) buffer. This brings to light that small aggregates form more readily in the former than in the latter buffer kind. As all the compared buffers ensured similar drug dissociation degree, this confirms the beneficial effect of buffering ions on the drug-loaded aggregates' adaptability in the final preparation. Drug ionisation using an alkaline pH and counterion hydrophobicity (to the extent differentiable with the chosen blend) may also be desirable. (Differences between ketoprofen- and indomethacin-containing mixed amphipat aggregates do exist: unlike the former, some of the latter are viscous at high pH, e.g. pH>8, especially the multicharged lysine and the bulky TRIS=tromethamine.)

Suspension characteristics of yet another NSAID, ketorolac (MW 255.27; log Pₘₑₘ^{N}˜2.3, log Pₘₑₘ^{I}˜0.8; pK=3.5; PK_{a,mem}˜5.5, best estimates), resembles ketoprofen in TRIS buffer around neutral pH. This suggests that higher polarity of the former NSAID is compensated by the more complete ionisation of the latter drug. Other qualitative conclusions made for ketoprofen also apply to ketorolac.

NSAID combinations with various non-phospholipid amphipats (or amphipat mixtures) of the BL-class, as defined herein, produce broadly similar experimental findings. Some sensitivity to Ac diversity is evident, however, e.g., polysorbate (=Tween) 21 (˜tetraoxyethylene sorbitan lauryl ester, Ac˜0.41 nm²) in a 0.1M phosphate buffer (pH≧7.7) separates in two phases, yielding a homogeneous, yet very turbid, suspension after short ultrasonication in 0.1 M meglumine buffer. The same amphipat/drug combination (7/3 w/w) affords an opalescent, straw yellow suspension after vortex mixing in 0.1 M TRIS buffer. In a 0.1 M epolamine buffer, a similar ketoprofen/Tween 21 blend yields an optically clear solution after mixing. A blend of 30 wt.-% ketoprofen in a less polar fatty-polyglyceride (Emulsogen OG, Ac˜0.31 nm²; total amphipat concentration 10 wt.-%) suspended in 0.1 M phosphate buffer behaves essentially like a suspension of 95% pure soybean phosphatidylcholine. However, replacing phosphate with tricine, TRIS, bicine or epolamine clearly makes the aggregates more adaptable, as is reflected in shorter vesicularisation times of the resulting suspensions (cf. Table 4).

Additional experimental data collected with sorbitan-alkylate and polyoxyethylene-sorbitan-alkyl-ether mixtures of the BL-type in combination with the other NSAIDs suspended in different organic or inorganic buffers support the conclusions. Sorbitan-alkylate and polyoxyethylene-sorbitan-alkylate mixtures that yield highly deformable and stable vesicles with an Ac near the upper vesicle stability-limit in absence of NSAIDs form a clear (i.e. solubilised) micellar suspension after NSAID addition. To make stable vesicles from the amphipat-NSAID combinations, one must therefore lower the relative concentration of polyoxyethylene-sorbitan-alkylate.

6.4 Aggregates Containing Positive Amphiphiles (e.g. Antifungal Terbinafine)

Table 5 highlights the related effects of organic salts, predominantly acids, on phosphatidylcholine vesicularisation kinetics with terbinafine (MW=291.43; pK=7.05; log Pₘₑₘ^{N}=5.19, log Pₘₑₘ^{I}=3.78; pK_{a,mem}=5.5, all pertaining to T=310 K and ˜10 w-% phospholipid plus drug concentration). The relatively high lipophilicity of terbinafine, which is cationic at low pH<<pK_{a,mem}, lessens this drug's impact on bilayer flexibility and vesicle adaptability compared to the tested NSAIDs. For example, in 0.1 M phosphate buffer at pH=5.7 (where a simple calculation suggests around 30% of terbinafine to be ionised), even a sonication time exceeding 1400 s fails to produce small aggregates above the initially formed precipitate. At pH=5.2 (simply calculated ionisation degree˜60%), the time needed to prepare drug-loaded aggregates in phosphate buffer is around 700 s. At pH˜4.4, where according to the simplest titration equation around 90% of terbinafine molecules should be charged, vesicularisation time of the preparations with 10 wt.-% total amphipat including 20 rel-% of the drug in phosphate buffer exceeds 200 s.

Replacing inorganic buffer with a suitable organic accelerates vesicularisation and significantly improves adaptability of the vesicles loaded with the partially anionic terbinafine, as is evident from Table 5. Non-cyclic ionic additives meet these goals better than cyclic ions, the relatively less bulky amongst the latter being relatively more attractive than the more bulky ones. The representative data given in Table 5 suggest that the bulkiest and the most lipophilic cyclic organic additives even stiffened the mixed amphipat bilayers when terbinafine was largely uncharged. Additives interacting with bilayers are thus useful only in a select range of distribution ratios.

The data provided in Tables 4 and 5 suggest that this sensitivity may be smaller, or even absent, for the moderately lipophilic, aggregate-bound charged molecules and/or for the systems with a thick bilayer-polar solvent interphase. The experimental data also suggests that relatively dilute hydrophilic (log P<0) buffers are more effective for purposes of this invention. In contrast, using less concentrated but relatively lipophilic buffers (such as azaleic acid, with log P>1) may bring no extra benefit (cf. Table 6), except in terms of its pKₐ. In the extreme, concentration lowering may lead to solubility problems or even competition for binding sites in aggregate bilayer.

In preparing the compositions, the additives will preferably have a distribution ratio at the chosen pH in the range −1±3, more preferably −1±2.5, and most preferably around −1±2. Optimally, several such additives will be tested in parallel at the outset, with the goal of confirming that the studied system optimum is at log P≦0 (as exemplified by ketoprofen-phosphatidylcholine mixtures at pH>6 or by terbinafine-phosphatidylcholine mixtures at pH˜3.6) or else profits from an increasing partition ratio (log P) and distribution ratio (e.g., to 0≦log D≦2, as exemplified by ketoprofen-Emulsogen-polysorbate mixture at pH˜6.2 and by terbinafine-phosphatidylcholine mixtures at pH˜4.8).

Table 7 shows several candidate additives and their preferred concentrations in the preparations of the invention. It does not specify the preferred thickener concentration, which is defined by specifying the resulting final product viscosity. The latter should preferably be between around 0.05 Pa s and around 10 Pa s, preferably between around 0.15 Pa s and around 5 Pa s, and most preferred between around 0.3 Pa s and about 2.5 Pa s for semisolid preparations. Thickener concentrations meeting this goal are in some embodiments typically chosen in the range from about 0.25 w-% to about 5 w-% relative to the total preparation weight, and preferably range from about 0.5 w-% to about 2.5 w-%. Most of commercially available carbopols can be used advantageously at concentrations around 1.5±0.75 wt.-%.

Collectively, the embodiments summarised in Tables 1-7 are useful in the selection of suitable (organic) additives (buffers), microbicide(s), antioxidant(s), etc.) with the aim of improving the vesicular aggregates adaptability. A plethora of amphipathic molecules, biological agents, (buffering) salts, and other additives can advantageously promote deformability of such drug-loaded aggregates, and thus beneficially improve the delivery and/or efficiency of the aggregate-associated drug activity after its application to a mammal. A non-limiting selection of the possible functional additives is provided in Table 7. The common goal is to achieve sufficiently high aggregate adaptability and/or payload, at least at the site and time of application of the aggregate preparation, including the option of a non-occlusive application on skin of such aggregates that are originally relatively rigid but then soften, and consequently become sufficiently adaptable to mediate the desired biological drug action, following an up-concentration of non-volatile preparation ingredients on an open skin surface. The preparations for parenteral delivery, on the other hand, will have to factor-in component dilution after an injection.

TABLE 1
Effect of pH and buffer selection on vesicularisation time of soybean
phosphatidylcholine in an aqueous suspension (bold pH values define the value at
which the phospholipid headgroups are zwitterionic)

| Nr. | Buffer/salt (total ionic strength: 100 mM) | pK | pH | t_{vesicle} [s] | tᵣₑₗ |
|---|---|---|---|---|---|
| 1 | Hydrochloric acid, sodium | −9.3 | 2.2 | 800 | 0.63 |
| 2 | Phosphoric acid, sodium | 2.1; 7.2, 12.4 | 3.3 | 1000 | 0.78 |
| 3 | Hydrochloric acid/NaCl | −9.3 | 4.3 | 1200 | 0.94 |
| 4 | Phosphoric acid, sodium | 2.1; 7.2, 12.4 | 5.6 | 1420 | 1.11 |
| 5 | Phosphoric acid, sodium | 2.1; 7.2, 12.4 | 5.6 | 1400 | 1.09 |
| 6 | Phosphoric acid, (di)sodium | 2.1; 7.2, 12.4 | 7.5 | 1200 | 0.94 |
| 7 | Phosphoric acid, disodium | 2.1; 7.2, 12.4 | 8.5 | 1175 | 0.92 |
| 8 | Sodium hydroxyde, NaCl | 13.0 | 12.5 | 950 | 0.74 |

| | | | Average neutral | | 1279 ± 120 | 1.00 ± 0.09 |
|---|---|---|---|---|---|---|
| 9 | | p-Toluenesulfonic acid (tosylic acid), sodium | 0.7 | 2.2 | 750 | 0.61 |
| 10 | | Gluconic acid | 3.6 | 2.5 | 650 | 0.53 |
| 11 | | p-Toluenesulfonic acid (tosylic acid), sodium | 0.7 | 4.0 | 950 | 0.77 |
| 12 | | Gluconic acid, sodium | 3.6 | 4.4 | 1200 | 0.98 |
| 13 | | Acetic acid | 4.8 | 5.0 | 1050 | 0.85 |
| 14 | | Pyroglutamic acid (pidolic acid) | 3.5 | 5.4 | 1500 | 1.22 |
| 15 | | Tetrabutylammonium bromide | | 6.6 | 250 | 0.20 |
| 16 | | Tromethamine (Tris) | 8.1 | 7.1 | 1200 | 0.98 |
| 17 | | Imidazole | 6.9 | 9.0 | 110 | 0.09 |
| 18 | | Morpholinoethanol | 7.4 | 8.0 | 1200 | 0.98 |
| 19 | | Morpholinoethanol | 7.4 | 9.0 | 80 | 0.07 |
| 20 | 1 | N-Methyl-D-glucamine (meglumine) | 9.5 | 9.4 | 200 | 0.16 |
| 21 | | Tromethamine (Tris) | 8.1 | 10.2 | 180 | 0.15 |
| 22 | | 1-Pyrrolidineethanol (epolamine) | 9.8 | 11.0 | 100 | 0.08 |

| | | |
|---|---|---|
| Average neutral | 1230 ± 164 | 1.00 ± 0.13 |
| Average neutral | 1231 ± 152 | 1.00 ± 0.12 |

TABLE 2
Vesicularisation time (t_{vesicle}) of various nonionic amphipat blends as a function of mixed aggregate composition,
relative molar ratio (M1 + M2 or M1/M2), the resulting average area per hydrophobic chain, Ac, polarity units number, nP, or the
corresponding HLB number.
(The number following C18 indicates the average nominal number of double bonds per hydrophobic chain; n₁ and n₂ the nominal
number of such chains per amphipat, and EG/n₁ and EG/n₁, or nG, the number of polar segments per headgroup (for
glycerophosphatides translated into nEO.)

| | | | | | | | | | | | | | | | | | | | | | | | |
|---|---|---|---|---|---|---|---|---|---|---|---|---|---|---|---|---|---|---|---|---|---|---|---|
| Column | 1 | 2 | 3 | 4 | 5 | 6 | 7 | 8 | 9 | 11 | 12 | 13 | 14 | 15 | 16 | 17 | 18 | 19 | 20 | 21 | 22 | 23 | 24 |

| | | | | | | |
|---|---|---|---|---|---|---|
| Amphipat characteristics Amphipats | Aggregate compositions | Mol. weight [symbol] | Ac | nP | HLB | t_{vesicle} [s] |

| | | | | |
|---|---|---|---|---|
| Polyalcohol derivatives | 1 | 2 | 1 | 2 |

| | | | | | | |
|---|---|---|---|---|---|---|
| Chains length, number, type | Head | Head | #1/#2 | #1/#2 | #1/#2 | pH |

| | | | | | | | | | | | |
|---|---|---|---|---|---|---|---|---|---|---|---|
| Emb. | L₁ | DB₁ | n₁ | n₂ | L₂ | DB₂ | w + w | M + M | M/M | MW1 | MW2 |

| Nr. | Polyoxyethyleneglycol (pEG) | | | | | Bond type | | EG/n₁ | EG/n₂ | | | g L⁻¹ | g L^{-1 Xn1} | | Xn2^{b} | | | | Mwaver | | | | | |
|---|---|---|---|---|---|---|---|---|---|---|---|---|---|---|---|---|---|---|---|---|---|---|---|---|
| 23 | C 18:1 | 1 | 1 | C18 | :1 | ET | ET | 0.0 | 10.0 | Oleyl | Brij 97 | 30.0 | 70.00 | 0.53 | 0.47 | 1.13 | 1 | 7 | 270 | 710 | 0.36 | 4.7 | 10.1 | long |
| | | | | | | | | | | alcohol | | | | | | | | | | | | | | |
| 24 | C 18:1 | 1 | 1 | C18 | :1 | ET | ET | 0.0 | 10.0 | Oleyl | Brij 97 | 24.0 | 76.00 | 0.45 | 0.55 | 0.83 | 1 | 7 | 270 | 710 | 0.39 | 5.5 | 10.4 | 360 |
| | | | | | | | | | | alcohol | | | | | | | | | | | | | | |
| 25 | C 18:1 | 1 | 1 | C18 | :1 | ET | ET | −0.5 | 10.0 | Oleic acid | Brij 97 | 24.0 | 76.00 | 0.47 | 0.53 | 0.9 | 1 | 7 | 248 | 710 | 0.38 | 4.8 | 8.9 | >600 |
| 26 | C 18:1 | 1 | 1 | C18 | :1 | ES | ES | 4.0 | 13.0 | C18:1EO4 | C18:1EO13 | 65.0 | 35.00 | 0.78 | 0.22 | 3.46 | 1 | 7 | 458 | 854 | 0.45 | 5.5 | 9.0 | >450 |
| | indirect aliphatic chain(s) attachment | | | | | | | | | | | | | | | | | | | | | | | |
| | sorbitan, branched headgroup; stochastic aliphatic (“anchor”) chains distribution | | | | | | | | | | | | | | | | | | | | | | | |
| 27 | C 18:1 | 1 | 1 | C18 | :1 | SES | SES | 0.0 | 20.0 | Span 80 | Tween 80 | 35.0 | 65.00 | 0.62 | 0.38 | 1.6 | 1 | 7 | 431 | 1311 | 0.34 | 4.5 | 8.4 | 900 |
| Polyglyceryl (pG) nG nG/nEO | | | | | | | | | | | | | | | | | | | | | | | | |
| | multiple aliphatic “anchor” chain attachment options/(stochastic) distribution | | | | | | | | | | | | | | | | | | | | | | | |
| 28 | C 18:1 | 1 | 1 | C18 | :1 | ES-G | SES | 2.0 | 20.0 | Emulsogen | Tween 80 | 70.0 | 30.00 | 0.87 | 0.13 | 6.83 | 1 | 7 | 448 | 1311 | 0.29 | 1.8 | 8.9 | >1200 |
| | | | | | | | | | | OG: [symbol] | | | | | | | | | | | | | | |
| 29 | C 18:1 | 1 | 1 | C18 | :1 | ES-G | ES-G | 2.0 | 5.0 | Emulsogen | Dermofeel | 15.0 | 85.00 | 0.22 | 0.78 | 0.28 | 1 | 7 | 448 | 718 | 0.46 | 2.4 | 13.5 | 420 |
| | | | | | | | | | | OG: [symbol] | G 5O | | | | | | | | | | | | | |
| 30 | C 18:1 | 1.5 | 1 | C18 | :1 | ES-G | | 10.0 | | Caprol | | 100.0 | 0.00 | 1.00 | 0.00 | 9999 | 1 | 7 | 1302 | | 0.33 | 1.1 | 11.0 | 780 (flaky [symbol] |
| | | | | | | | | | | PGE 860 | | | | | | | | | | | | | | |
| (Poly)Sugar derivatives (here, (poly)hexose nₕₑₓ nₕₑₓ | | | | | | | | | | | | | | | | | | | | | | | | |
| | multiple “anchor” chain attachment options/(stochastic) distribution | | | | | | | | | | | | | | | | | | | | | | | |
| 31 | C 18:1 | 5 | 1 | C18 | :1 | ES | ES | 0.4 | 1.7 | Surfhope | Surfhope | 75.0 | 25.00 | 0.55 | 0.45 | 1.24 | | 7 | 1517 | 628 | 0.40 | 3.7 | 7.2 | long |
| | | | | | | | | | | C-1701 | C-1715 | | | | | | | | | | | | | |
| | Glycerophosphatides P-head^{c} EO | | | | | | | | | | | | | | | | | | | | | | | |
| 32 | C 18:2 | 2 | 1 | | | ES | | 4.0 | | SPC | | 100.0 | 0.0 | 1.00 | 0.00 | 9999 | 1 | 7 | 810 | 1150 | 0.35 | 2.0 | 8.0 | 1200 |
| 33 | C 18:2 | 2 | 1 | C18 | :1 | ES | ET | 4.0 | 20.0 | SPC | Brij 98 | 87.57 | 12.4 | 0.91 | 0.09 | 10.00 | 1 | 7 | 810 | 1150 | 0.38 | 3.6 | 8.7 | 330 |

[symbol]

indicates data missing or illegible when filed

TABLE 3
Vesicularisation time (t_{vesicle}) of various nonionic amphipat blends as a function of mixed aggregate composition,
relative molar ratio (M1 + M2 or M1/M2), the resulting average area per hydrophobic chain, Ac, or the corresponding polarity units
count, nP, or HLB number. (L_{1,2} or L₂ defines the 1^{st} or 2^{nd} and 2^{nd} aliphatic chain length, the number after C18: gives the average
nominal number of double bonds per such chain; n₁ and n₂ the nominal number of hydrophobic chains per amphipat; and EG/n₁ and
EG/n₁ or nG the number of polar segments per headgroup (in the case of glycerophosphatides translated into the EO-analogy.)

| | | | | | | | | | | | | | | | | | | | | | | | |
|---|---|---|---|---|---|---|---|---|---|---|---|---|---|---|---|---|---|---|---|---|---|---|---|
| Colmn | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 |

| | | | | | |
|---|---|---|---|---|---|
| Amphipat characteristics Amphipats | Aggregate compositions Mol. whts | Ac | nP | HLB | tᵥₑₛ [s] |

| | | | | |
|---|---|---|---|---|
| Polyalcohol derivatives | 1 | 2 | 1 | 2 |

| | | | | | | |
|---|---|---|---|---|---|---|
| Chains length, number, type | Head | Head | #1/#2 | #1/#2 | #1/#2 | pH |

| | | | | | | | | | | | |
|---|---|---|---|---|---|---|---|---|---|---|---|
| Emb. | L₁ | DB₁ | n₁ | n₂ | L₂ | DB₂* | w + w | M + M | M/M | MW1 | MW2 |

| Nr. | Polyoxyethyleneglycol (pEG) | | | | | Bond typ [symbol] | | EG/n₁ | EG/n₂ | | | g L⁻¹ | g L⁻¹ | Xn1 | Xn2^{b} | | | | Mwaver | | | | | |
|---|---|---|---|---|---|---|---|---|---|---|---|---|---|---|---|---|---|---|---|---|---|---|---|---|
| | direct aliphatic chains attachment*, linea headgroup | | | | | | | | | | | | | | | | | | | | | | | |
| 34 | C 12 | 1 | 0 | | | ET | | 4.0 | | Brij30 | | 100.0 | 0.00 | 1.00 | 0.00 | 9999 | 1 | 7.4 | 362 | 0 | 0.42 | 4.0 | 9.6 | 5 |
| 35 | C 18:1 | 1 | 1 | | | ET | ET | 2.0 | 10.0 | Brij93 | Brij 97 | 29.0 | 71.00 | 0.45 | 0.55 | 0.81 | 1 | 7.4 | 358 | 710 | 0.47 | 6.4 | 9.0 | 55 |
| 36 | C 18:1 | 1 | 1 | | | ES | ES | 4.0 | 13.0 | C18:1EO4 | C18:1EO13 | 40.0 | 60.00 | 0.55 | 0.45 | 1.24 | 1 | 7.4 | 458 | 854 | 0.48 | 7.5 | 8.4 | 10 |
| 37 | C 18:1 | 1 | 0 | | | ET | | 5.5 | | Emulsogen | | 100.0 | 0.00 | 1.00 | 0.00 | 9999 | 1 | 7.4 | 512 | 0 | 0.46 | 5.5 | 9.3 | 20 |
| | | | | | | | | | | LP | | | | | | | | | | | | | | |
| | indirect aliphatic chain(s) attachment | | | | | | | | | | | | | | | | | | | | | | | |
| | sorbitan, branched headgroup; stochastic aliphatic (“anchor”) chains distribution | | | | | | | | | | | | | | | | | | | | | | | |
| 38 | C 12 | 1 | 1 | | | SES | SES | 0.0 | 20.0 | Span 20 | Tween 20 | 50.0 | 50.00 | 0.78 | 0.22 | 3.5 | 1 | 7.4 | 347 | 1227 | 0.34 | 2.6 | 10.4 | |
| 39 | C 12 | 1 | 0 | | | SES | SES | 4.0 | | Tween 21 | | 100.0 | 0.00 | 1.00 | 0.00 | 9999 | 1 | 7.4 | 523 | | 0.40 | 2.4 | 11.0 | 15 |
| 40 | C 18:1 | 1 | 1 | | | SES | SES | 0.0 | 20.0 | Span 80 | Tween 80 | 17.5 | 82.50 | 0.39 | 0.61 | 0.6 | 1 | 7.4 | 431 | 1311 | 0.43 | 7.2 | 10.8 | 20 |
| 41 | C 18:1 | 1 | 1 | | | ET | SES | 2.0 | 20.0 | Brij93 | Tween 80 | 22.5 | 77.50 | 0.52 | 0.48 | 1.1 | 1 | 7.4 | 358 | 1311 | 0.45 | 6.3 | 9.5 | 30 |
| 42 | C 18:1 | 1 | 1 | | | SES | SES | 5.0 | 20.0 | Tween 81 | Tween 80 | 50.0 | 50.00 | 0.67 | 0.33 | 2.0 | 1 | 7.4 | 651 | 1311 | 0.45 | 5.9 | 11.7 | 25 |
| 43 | C 18:1 | 3 | 1 | | | SES | SES | 20.0 | 20.0 | Tween 85 | Tween 80 | 85.0 | 15.00 | 0.80 | 0.20 | 4.0 | 1 | 7.4 | 1851 | 1311 | 0.45 | 5.5 | 11.0 | 20 |
| Polyglyceryl (pG) nG nG/nEG | | | | | | | | | | | | | | | | | | | | | | | | |
| | multiple aliphatic “anchor” chain attachment options/(stochastic) distribution | | | | | | | | | | | | | | | | | | | | | | | |
| 44 | C 12 | 1 | 1 | C18 | :1 | ES-G | (ET) | 5.0 | 0.0 | Dermofeel | Octa- | 90.0 | 10.00 | 0.79 | 0.21 | 3.83 | 1 | 7.4 | 634 | 270 | 0.45 | 6.5 | 12.1 | 10 |
| | | | | | | | | | | G 5 L | decanol | | | | | | | | | | | | | |
| 45 | C 18:1 | 1.6 | | | | ES-G | ES-G | 5.0 | | Dermofeel | | 100.0 | 0.00 | 1.00 | 0.00 | 9999 | 1 | 7.4 | 882 | | 0.45 | 5.1 | 8.0 | 15 |
| | | | | | | | | | | G 5 DO | | | | | | | | | | | | | | |
| 46 | C 18:1 | 1 | 1 | | | ES-G | SES | 2.0 | 20.0 | Emsulsogen | Tween 80 | 25.0 | 75.00 | 0.49 | 0.51 | 0.98 | 1 | 7.4 | 448 | 1311 | 0.42 | 6.1 | 11.5 | 90 |
| | | | | | | | | | | OG: Em | | | | | | | | | | | | | | |
| 47 | C 18:1 | 1 | 1 | | | ES-G | SES | 2.0 | 20.0 | Emsulsogen | Tween 80 | 15.0 | 85.00 | 0.34 | 0.66 | 0.52 | 1 | 7.4 | 448 | 1311 | 0.47 | 7.9 | 12.6 | 50 |
| | | | | | | | | | | OG: Em | | | | | | | | | | | | | | |
| 48 | C 18:1 | 1.5 | 1 | | | ES-G | SES | 10.0 | 20.0 | Caprol | Tween 80 | 50.0 | 50.00 | 0.50 | 0.50 | 1.01 | 1 | 7.4 | 1302 | 1311 | 0.45 | 6.4 | 13.0 | 30 |
| | | | | | | | | | | PGE 860 | | | | | | | | | | | | | | |
| | (Poly)Sugar derivatives (here, (poly)hexose) | | | | | | | | | | | | | | | | | | | | | | | |
| nₕₑₓ nₕₑₓ | | | | | | | | | | | | | | | | | | | | | | | | |
| | multiple “anchor” chain attachment options/(stochastic) distribution | | | | | | | | | | | | | | | | | | | | | | | |
| 49 | C 18:1 | 5 | 1 | C18 | :1 | ES | ES | 0.4 | 1.7 | Surfhope | Surfhope | 50.0 | 50.00 | 0.29 | 0.71 | 0.41 | | 7.4 | 1517 | 628 | 0.44 | 5.0 | 10.9 | short |
| | | | | | | | | | | C-1701 | C-1715 | | | | | | | | | | | | | |
| 50 | C 18:1 | 5 | 1 | C18 | :1 | ES | ES | 0.4 | 1.7 | Surfhope | Surfhope | 25.0 | 75.00 | 0.12 | 0.88 | 0.14 | | 7.4 | 1517 | 628 | 0.47 | 5.9 | 13.3 | short |
| | | | | | | | | | | C-1701 | C-1715 | | | | | | | | | | | | | |
| | Glycerophosphatides P-head^{c} EO | | | | | | | | | | | | | | | | | | | | | | | |
| 51 | C 18:2 | 2 | 1 | | :1 | ES | ET | 4.0 | 20.0 | SPC | Brij 98 | 73.80 | 26.2 | 0.80 | 0.20 | 4.00 | 1 | 7.4 | 810 | 1150 | 0.42 | 5.6 | 9.5 | 95 |
| 52 | C 18:2 | 2 | 1 | | :1 | ES | ET | 4.0 | 20.0 | SPC | Brij 98 | 67.88 | 32.1 | 0.75 | 0.25 | 3.00 | 1 | 7.4 | 810 | 1150 | 0.44 | 6.5 | 9.8 | 70 |
| 53 | C 18:2 | 2 | 1 | | :1 | ES | SES | 4.0 | 20.0 | SPC | Tween 80 | 60.70 | 39.3 | 0.71 | 0.29 | 2.50 | 1 | 7.4 | 810 | 1311 | 0.42 | 4.8 | 10.0 | 40 |

*ET = ether; ES = ester; SES = sorbitan-ester (with an unspecified proportion of non-esterified material); ES-G = ester of a polyglyceryl;
^{a}If not otherwise specified, all amphipats have similar chains, on the average. [symbol]
indicates data missing or illegible when filed

TABLE 4
Vesicularisation time (t_{vesicle}) and its relative value compared with the phosphate buffer reference value
(Average @ 6 < pH < 7.5), tᵣₑₗ, of various mixed aggregates loaded with ketoprofen as a function of hydrophilic additive and preparation pH.

| | Buffer/salt | | | | | | | Drug + Amphipat | | | | | | | | | | | | |
|---|---|---|---|---|---|---|---|---|---|---|---|---|---|---|---|---|---|---|---|---|
| | (total ionic | | | | | | | | | | KT + Em | | | | | | | | | |
| | strength: | | | | | | | KT + SPC | | | OG/Tw 80 | | | KT + Tw 21 | | | | IND + SPC | | |
| | 100 mM) | | | | | Buffer | | t_{vesicle} | | | | t_{vesicle} | | | t_{vesicle} | | | | t_{vesicle} | |
| Nr. | Trivial name | pK1 | pK2 | pK3 | pH | Charge | log D [symbol] | [s] | tᵣₑₗ | Nr. | pH | [s] | tᵣₑₗ | pH | [s] | tᵣₑₗ | Nr. | pH | [s] | tᵣₑₗ |
| 54 | Sodium | | | | 3.3 | | | Precipitate | | | | | | | | | | | | |
| | chloride | | | | | | | | | | | | | | | | | | | |
| 55 | Sodium | | | | 6.0 | | | 40 | 0.9 | | | | | | | | | | | |
| | chloride/ | | | | | | | | | | | | | | | | | | | |
| | hydroxide | | | | | | | | | | | | | | | | | | | |
| 56 | Sodium | | | | 6.7 | | | 40 | 0.9 | | | | | | | | | | | |
| | chloride/ | | | | | | | | | | | | | | | | | | | |
| | hydroxide | | | | | | | | | | | | | | | | | | | |
| 57 | Phosphoric | 2.1 | 7.2 | | 3.8 | −1.0 | −2.9 | Precipitate | | | | | | | | | | | | |
| | acid | | | | | | | | | | | | | | | | | | | |
| 58 | Phosphoric | 2.1 | 7.2 | | 5.9 | −1.0 | −2.9 | 150 | | | | | | | | | | | | |
| | acid | | | | | | | | | | | | | | | | | | | |
| 59 | Phosphoric | 2.1 | 7.2 | | 6.2 | −1.1 | −2.9 | 50 | 1.2 | 82 | 5.8 | 1200 | 1.0 | | 2Phase | | 92 | 6.7 | 200 | 1.0 |
| | acid | | | | | | | | | | | | | | | | | | | |
| 60 | Phosphoric | 2.1 | 7.2 | | 7.2 | −1.5 | −3.1 | 40 | 0.9 | | | | | | | | | | | |
| | acid | | | | | | | | | | | | | | | | | | | |
| 61 | Sulphuric acid | −3 | 1.9 | | 6.2 | −2.0 | −3.5 | 120 | | | | | | | | | | | | |
| 62 | Sulphuric acid | −3 | 1.9 | | 6.7 | −2.0 | −3.5 | 95 | 2.2 | | | | | | | | 93 | 6.5 | 250 | 1.3 |
| 63 | Sulphuric acid | −3 | 1.9 | | 7.2 | −2.0 | −3.5 | 40 | 0.9 | | | | | | | | | | | |
| 64 | Sulphuric acid | −3 | 1.9 | | 8.8 | −2.0 | −3.5 | 20 | | | | | | | | | | | | |
| | | | | Average @ 6 < pH < 7.5 | | | | 43 ± 33 | | | | 1200 | | | | | | | 200 | |
| 65 | PIPES | 7.1 | 0.4 | | 5.7 | 0.0 | −3.5 | 45 | | | | | | | | | | | | |
| 66 | PIPES | 7.1 | 0.4 | | 6.5 | 0.2 | −3.7 | 25 | 0.6 | | | | | | | | | | | |
| 67 | Meglumine | 9.5 | | | 6.1 | 1.0 | −3.7 | 50 | 1.2 | 83 | 6.4 | 600 | 0.5 | | Turbid | | 94 | 8.6 | 30 | 0.2 |
| 68 | Lysine | 10.5 | 9.1 | 2.2 | 6.3 | 1.0 | −3.5 | 60 | 1.4 | | | | | | | | 95 | 6.8 | 100 | 0.5 |
| 69 | Bis-TRIS | 6.5 | | | 6.1 | 0.7 | −3.4 | 60 | 1.4 | | | | | | | | | | | |
| 70 | Tricine | 8.2 | 2.3 | | 4.9 | 0.0 | −2.9 | Precipitate | | | | | | | | | | | | |
| 71 | Tricine | 8.2 | 2.3 | | 6.2 | 0.0 | −2.9 | 20 | 0.5 | 84 | | 600 | | | | | | | | |
| 72 | TRIS | 8.1 | | | 6.3 | 1.0 | −3.1 | 30 | 0.7 | 85 | 6.3 | 400 | 0.3 | | Opalescent | | | | | |
| 73 | TRIS | 8.1 | | | 7.7 | 0.7 | −2.8 | 10 | 0.2 | | | | | | | | 96 | 8.0 | 20 | 0.1 |
| 74 | HEPES | 7.6 | 1.7 | | 6.5 | 0.0 | −2.5 | 120 | 2.8 | | | | | | | | | | | |
| 75 | Choline, | 13.9 | | | 6.5 | 1.0 | −2.4 | Precipitate | | | | | | | | | | | | |
| | chloride | | | | | | | | | | | | | | | | | | | |
| 76 | Bicine | 8.5 | 1.9 | | 6.2 | 0.0 | −2.1 | 20 | 0.5 | | | | | | | | | | | |
| 77 | Imidazole | 14.5 | 6.9 | | 6.0 | 1.9 | −2.0 | 20 | 0.5 | | | | | | | | | | | |
| 78 | Morpholino- | 7.4 | | | 6.1 | 1.0 | −1.6 | 20 | 0.5 | 86 | | 280 | | | | | 97 | 7.3 | 30 | 0.2 |
| | ethanol | | | | | | | | | | | | | | | | | | | |
| 79 | Epolamine | 9.8 | | | 6.2 | 1.0 | −0.8 | 20 | 0.5 | 87 | 6.3 | 200 | 0.2 | | Opalescent/ | | 98 | 7.8 | 30 | 0.2 |
| | | | | | | | | | | | | | | | clear | | | | | |
| 80 | Diethylamine | 10.6 | | | 6.2 | 1.0 | −0.3 | 45 | 1.1 | | | | | | | | | | | |
| 81 | Tetrabutyl- | | | | 6.3 | 1.0 | 3.8 | Precipitate | | | | | | | | | | | | |
| | ammonium, | | | | | | | | | | | | | | | | | | | |
| | bromide | | | | | | | | | | | | | | | | | | | |

[symbol]

indicates data missing or illegible when filed

TABLE 5
Vesicularisation time (t_{vesicle}) and its relative value compared with the phosphate buffer reference value
(Average @ 6 < pH < 7.5), tᵣₑₗ, of various mixed aggregates loaded with terbinafine as a function of hydrophilic additive and preparation pH.

| | Buffer/salt | | | | | | | | | | | | | | | |
|---|---|---|---|---|---|---|---|---|---|---|---|---|---|---|---|---|
| | (total | | | | | | | Drug + Amphipat | | | | | | | | |
| | ionic strength: | | | | | | | | | | TBF + | | | | | |
| | 100 mM) | | | | | Buffer | | TBF + SPC | | | Em OG/Tw 80 | | | | TBF + Tw 21 | |
| Nr. | Trivial name | pK1 | pK2 | pK3 | pH | Charge | log D_{calc} | t_{vesicle} [s] | t_{ref} | Nr. | pH | t_{vesicle} [s] | t_{ref} | Nr. | pH | t_{vesicle} [s] |
| 99 | Sodium chloride | | | | 2.6 | | | 80 | | | | | | | | |
| | (NaCl) | | | | | | | | | | | | | | | |
| 100 | HCl/NaCl | −9.3 | | | 2.5 | | | 190 | | | | | | | | |
| 101 | Phosphoric acid, | 2.2 | 7.2 | | 5.7 | −1.0 | −2.9 | Precipitate | | | | | | | | |
| | sodium | | | | | | | | | | | | | | | |
| 102 | Phosphoric acid, | 2.2 | 7.2 | | 5.2 | −1.0 | −2.9 | 700 | | | | | | | | |
| | sodium | | | | | | | | | | | | | | | |
| 103 | Phosphoric acid, | 2.2 | 7.2 | | 4.4 | −1.0 | −2.9 | 225 | 1.00 | | | | | | | |
| | sodium | | | | | | | | | | | | | | | |
| 104 | Phosphoric acid, | 2.2 | 7.2 | | 3.7 | −1.0 | −2.9 | 150 | 1.00 | | | | | | | |
| | sodium | | | | | | | | | | | | | | | |
| 105 | Phosphoric acid, | 2.2 | 7.2 | | 3.3 | −0.9 | −2.8 | 120 | | | | | | | | |
| | sodium | | | | | | | | | | | | | | | |
| 106 | Phosphoric acid, | 2.2 | 7.2 | | 3.1 | −0.9 | −2.8 | 100 | | | | | | | | |
| | sodium | | | | | | | | | | | | | | | |
| 107 | Phosphoric acid | 2.2 | 7.2 | | 1.0 | −0.1 | −2.0 | 60 | | | | | | | | |
| 108 | Glycerol-2- | 1.3 | 7.0 | | 3.0 | −1.0 | −3.5 | 120 | | | | | | | | |
| | phosphate | | | | | | | | | | | | | | | |
| 109 | Gluconic acid | 3.6 | | | 3.7 | −0.6 | −3.4 | 110 | 0.73 | | | | | 149 | | Opalescent |
| 110 | Gluconic acid | 3.6 | | | 3.5 | −0.4 | −3.2 | 120 | 0.80 | | | | | | | |
| 111 | Gluconic acid | 3.6 | | | 2.4 | −0.1 | −2.9 | 50 | | | | | | | | |
| 112 | Gluconic acid | 3.6 | | | 2.3 | 0.0 | −2.8 | 50 | | | | | | | | |
| 113 | Glucuronic acid | 3.6 | | | 3.7 | −0.6 | −3.2 | 140 | 0.93 | 147 | | 50 | | 150 | | Opalescent |
| 114 | Glucuronic acid | 3.6 | | | 3.8 | −0.6 | −3.2 | 100 | 0.67 | | | | | | | |
| 115 | Tartaric acid | 3.0 | 4.3 | | 4.2 | −1.4 | −3.2 | 270 | 1.20 | | | | | | | |
| 116 | Citric acid | 3.1 | 4.8 | 6.4 | 4.4 | −1.3 | −3.0 | 950 | 4.22 | | | | | | | |
| 117 | MES | 6.2 | | | 3.0 | 0.0 | −2.1 | 70 | | | | | | | | |
| 118 | Pyroglutamic | 3.5 | | | 5.7 | −1.0 | −2.1 | 1800 | | | | | | | | |
| | acid, | | | | | | | | | | | | | | | |
| | pidolic acid | | | | | | | | | | | | | | | |
| 119 | Pyroglutamic acid | 3.5 | | | 4.8 | −1.0 | −2.1 | 800 | | | | | | | | |
| 120 | Pyroglutamic acid | 3.5 | | | 3.4 | −0.4 | −1.5 | 50 | 0.33 | 148 | | 15 | | 151 | pH? Clear/iridescent | |
| 121 | Pyroglutamic acid | 3.5 | | | 2.0 | 0.0 | −1.1 | 30 | | | | | | | | |
| 122 | Methylmalonic; | 4.4 | 2.8 | | 4.6 | −1.6 | −1.9 | 480 | 2.13 | | | | | | | |
| | iso-succinic | | | | | | | | | | | | | | | |
| 123 | Besylic acid | 0.7 | | | 4.6 | −1.0 | −1.2 | 300 | 1.33 | | | | | | | |
| 124 | Besylic acid | 0.7 | | | 3.7 | −1.0 | −1.2 | 30 | 0.20 | | | | | | | |
| 125 | Tosyllic acid | 0.7 | | | 2.8 | −1.0 | −0.7 | 30 | | | | | | | | |
| 126 | Tosyllic acid | 0.7 | | | 3.2 | −1.0 | −0.7 | 30 | | | | | | | | |
| 127 | Tosyllic acid | 0.7 | | | 3.8 | −1.0 | −0.7 | 30 | 0.20 | | | | | 152 | | Opalescent |
| 128 | Acetic acid | 4.8 | | | 4.8 | −0.5 | −0.7 | 225 | | | | | | | | |
| 129 | Acetic acid | 4.8 | | | 4.4 | −0.3 | −0.5 | 140 | 0.62 | | | | | | | |

| | | | | | | | | | |
|---|---|---|---|---|---|---|---|---|---|
| 130 | Acetic acid | 4.8 | 3.8 | −0.1 | −0.2 | 75 | 0.50 | 153 | Opalescent/grainy |

| | | | | | | | | |
|---|---|---|---|---|---|---|---|---|
| 131 | Acetic acid | 4.8 | | 2.4 | 0.0 | −0.1 | 30 | |
| 132 | Adipic acid | 4.4 | 5.4 | 4.5 | −0.6 | −0.4 | 300 | 1.33 |
| 133 | Gallic acid | 4.4 | | 4.5 | −0.6 | 0.1 | 510 | 2.27 |
| 134 | Gallic acid | 4.4 | | 3.7 | −0.1 | 0.5 | 360 | 2.40 |
| 137 | MOPS | 7.3 | | 4.6 | 0.0 | 0.3 | 520 | 2.31 |
| 136 | MOPS | 7.3 | | 3.6 | 0.0 | 0.3 | 240 | 1.60 |
| 135 | MOPS | 7.3 | | 2.6 | 0.0 | 0.3 | 60 | |
| 138 | Methyl-adipic | 4.8 | | 3.8 | −0.1 | 1.0 | 130 | 0.87 |
| | acid | | | | | | | |
| 139 | Methyl-adipic | 4.8 | | 4.5 | −0.3 | 0.8 | 130 | 0.58 |
| | acid | | | | | | | |
| 140 | Hydroxybenzoic | 4.5 | | 4.5 | −0.5 | 0.8 | 390 | 1.73 |
| | acid; paraben | | | | | | | |
| 141 | Benzoic acid | 4.1 | | 4.4 | −0.7 | 1.1 | Precipitate | |
| 142 | Benzoic acid | 4.1 | | 2.3 | 0.0 | 1.7 | 30 | |
| 143 | Indoleacetic acid | 4.6 | | 3.7 | −0.1 | 0.5 | 200 | 1.33 |
| 144 | Azelaic acid | 4.5 | 5.3 | 5.1 | −1.2 | 0.5 | 210 | |
| 145 | Azelaic acid | 4.5 | 5.3 | 4.6 | −0.7 | 1.0 | 55 | 0.24 |
| 146 | Azelaic acid | 4.5 | 5.3 | 4.0 | −0.3 | 1.4 | 45 | |

TABLE 6
Vesicularisation time (t_{vesicle}) and relative duration of vesicularisation,
tᵣₑₗ, as a function of hydrophilic additive (buffer) concentration in
phosphatidylcholine vesicles loaded either with the (partially,
dependent on pH) charged anionic ketoprofen or partially charged
cationic terbinafine.

| | | | pH | t_{vesicle} [s] | tᵣₑₗ |
|---|---|---|---|---|---|
| | Composition/Buffer | | compared with | | |
| Nr. | concentration [mM] | MW | 100 mM | | |
| | SPC + ketoprofen | | | | |
| | (7 + 3 w % + w %) | | | | |
| | TRIS | 121.16 | | | |
| 154 | 100 diluted in suspension to 25 | | 6.9 | 0 | 0 |
| 155 | 25 | | 6.0 | 45 | |
| 156 | 25 | | 6.8 | 25 | 0.8 |
| 157 | 50 | | 6.9 | 25 | 0.8 |
| 158 | 50 | | 8.3 | 25 | 0.8 |
| 159 | 100 | | 6.3 | 30 | 0.9 |
| 160 | 100 | | 7.6 | 35 | 1.1 |
| 161 | 200 | | 7.9 | 180 | 5.5 |
| | SPC + ketoprofen | | | | |
| | (7 + 3 w % + w %) | | | | |
| | Bicine | 163.7 | | | |
| 162 | 100 | | 6.2 | 20 | 0.6 |
| 163 | 200 | | 6.3 | 45 | 1.4 |
| | SPC + terbinafine | | | | |
| | (8 + 2 w % + w %) | | | | |
| | Azelaic acid | 188.22 | | | |
| 164 | 100 diluted in suspension to 25 | | 3.8 | 40 | |
| 165 | 25 | | 4.6 | 250 | 4.5 |
| 166 | saturated at RO | | 4.6 | 60 | 1.1 |
| 167 | ~100 | | 4.6 | 50 | 0.9 |

Collectively, the invention discloses a variety of amphipat combinations, not just phospholipid-surfactant blends, that form improved vesicular aggregates according to the selection and processing described herein, whereby the aggregate compositions are advantageously more adaptable and/or have an increased drug payload capacity and/or are more stable than known aggregates lacking the additives of the invention. The findings and teachings of the invention are thus useful for, but not limited to, manufacturing aggregate suspensions, dispersions, nanoemulsions, or microemulsions; improving such preparations' stability; use of the resulting preparations for agent(s) solubilisation, stabilisation, and/or application; overcoming transport barriers (e.g. in the course of aggregate filtration, microfiltration or nanofiltration); for aggregate-targeted, -mediated, -facilitated or -enhanced delivery of agents (e.g. through a nano- or micro-porous permeability barrier, such as skin, mucosa, cornea, etc.).

TABLE 7
Non-limiting list of various described additives included in the preparations
of the invention with specific and/or preferred concentration ranges.

| | | w [% rel.] | [mM] | | | | | | | | | | | | | | | |
|---|---|---|---|---|---|---|---|---|---|---|---|---|---|---|---|---|---|---|
| | to aggregate m. | | | Concentrations used [gL-1] | | | | | | | | | | | | | | |
| Microbicide | | | | | | | | | | | | | | | | | | |
| Acetic acid | 0.1-0.5 | | 10-66* | | | | | | | | | | | | | | | 4.00 |
| Benzoic acid | 0.05-0.5 | 0.05-0.5 | | | | | | | | | | | | | | | | |
| Dehydroacetic acid | 0.2-1.0 | | 10-75* | | | | | | | | | | | | | | | |
| Formic acid | 0.1-0.5 | | 20-100* | | | | | | | | | | | | | | | |
| Propionic acid | 0.2 | 0.2 | 20-100 | | | | | | | | | | | | | | | |
| Sorbic acid | | 0.025-0.1 | (5-25) | | | | | | | | | | | | | | | |
| Monomethylolglycine | 0.1-0.8** | | 10-80* | | | | | | | | | | | 5.00 | | | | |
| Dimethoxane | | 0.05-0.3 | | | | | | | | | | | | | | | | |
| 5-Bromo-5-nitro-1,3-dioxane, bronidox | 25-150 ml | 0.05-0.3 | (0.06-0.3) | 1.000 | | 0.05-0.3 | | | | | | | | | | | | |
| 2-bromo-2-nitropropane-1,3-diol, bronopol | 0.05-0.3 | | 25-150 | | | | | | | | | | | | | | 1.00 | |
| Diazodinyl Urea (Germall II) | 0.2-0.5 | | 75-175 | | | | | | | 5.00 | 2.00 | | | | | | | |
| 1,3-Dimethylol-5,5-dimethylhydantoin | | 0.15-0.4 | (7.5-30) | | | | | | | | | | | | | 1.50 | | |
| Methyl 4-hydroxybenzoate; methylparabene | | 0.1-5 | | | 1.125 | | | | 0.45 | | 0.45 | | | | | | | |
| Ethyl 4-hydroxybenzoate; ethylparabene | | 0.1-3 | | | | | | | | | | | | | | | | |
| Propyl 4-hydroxybenzoate; propylparabene | | 0.1-1.5 | | | 1.125 | | | | 0.45 | | 0.45 | | | | | | | |
| Butyl 4-hydroxybenzoate; butylparabene | | 0.1-3 | | | 0.250 | | | | 0.10 | | 0.10 | | | | | | | |
| Methylisothiazolone | 0.05-0.1 | | 50-100 | | | 0.500 | 0.075 | | 0.01 | | | | | | | | | |
| Methylchloroisothiazolinone | | 0.05-0.1 | | | | | | 0.50 | | | | | | | | | | |
| Benzisothiazolone | | 0.05-0.25 | | | | | | | | | | | | | | | | |
| Phenoxyethanol | | 0.1-5 | | | | | | | | | | | | | 1.00 | | | |
| Phenylethanol | | 0.1-1 | | | | | | | | | | | | | | | | |
| Quaternium-15; Dow [symbol] cll ® 200 | 0.05-0.3 | | 20-125 | | | | | | | | | 2.00 | | | | | | |
| Nipaguard ® CMB or DCB or PBI | 0.03-0.3 | | 0.03-0.3** | | | | | | | | | | 0.015* | | | | | |
| Euxyl ® K 145 | 0.05-0.15 | | 0.05-0.15** | | | | | | | | | | 1.50 | | | | | |
| Co-solvent | | | | | | | | | | | | | | | | | | |
| Benzyl alcohol | | 0-0.5 | | | | | 5.000 | | | | | | | | | | | |
| Ethanol | | 0-15 | 0-15** | | | | | | | | | | | | | | | |
| Propanediol | | 0-15 | 0-10** | | | | | | | | | | | | | | | |
| Humectant | | | | | | | | | | | | | | | | | | |
| Glycerol | | | 0-15** | 5.00** | 10.00*** | | | | | | | | | | | | | |
| Urea | | | 0-15** | 5.00** | 10.00*** | | | | | | | | | | | | | |
| Triacetin, glycerin triacetate | | | 2.5** | 2.500 | | | | | | | | | | | | | | |
| Tetramethylurea | | | 0-10** | | | | | | | | | | | | | | | |
| Hyaluronic acid | | | 0-5** | | | | | | | | | | | | | | | |
| Antioxidant | | | | | | | | | | | | | | | | | | |
| 2,3,5-trimethylbenzene-1,4-diol | 0.300 | 0.1-10 | | | | | | | | | | | | | | | | |
| Butylated hydroxyanisole, BHA | 0.250 | 0.1-8 | | | | | | | | | | | | | | | | |
| 4-hexoxy-2,3,5-trimethylphenol, HTHQ | | 0.1-8 | | | | | | | | | | | | | | | | |
| Butylated hydroxytoluene, BHT | | 0.1-4 | | | | | | | | | | | | | | | | |
| 6-isopropyl-m-cresol; thymol | | 0.1-1 | | | | | | | | | | | | | | | | |
| Alkyl-ascorbate | | 0.1-1 | | | | | | | | | | | | | | | | |
| Tocopherol | | 0-5 | | | | | | | | | | | | | | | | |
| Tocopherol-PEG | | 0-5 | | | | | | | | | | | | | | | | |
| Ascorbic acid | | | 0-2** | | | | | | | | | | | | | | | |
| Metabisulphite | | | 1-5** | | | | | | | | | | | | | | | |
| Bisulphite | | | 1-5** | | | | | | | | | | | | | | | |
| Thiourea | | | 1-10** | | | | | | | | | | | | | | | |
| 3-sulfanylpropane-1,2-diol | | | 1-20** | | | | | | | | | | | | | | | |
| Chellator (secondary “antioxidant”) | | | | | | | | | | | | | | | | | | |
| Br-Benzyl-teta | 3.000 | | 1-8 | | | | | | | | | | | | | | | |
| Edetic acid (EDTA | | | 1-10 | | | | | | | | | | | | | | | |
| Egtazic acid (EGTA) | | | 1-10 | | | | | | | | | | | | | | | |
| Ethyleneglycol-bis-N,N′-tetracetic acid | | | 1-10 | | | | | | | | | | | | | | | |
| Deferoxamine | | | 0.1-5 | | | | | | | | | | | | | | | |
| Fragrance | | | | | | | | | | | | | | | | | | |
| Linalool | 1.000 | | | 0.500 | | | | | | | | | | | | | | |
| Myrcene/myrcenol | | 0.500 | | | 0.250 | | | | | | | | | | | | | |
| Menthol | | | 0.250 | | | 0.500 | | | | | | | | | | | | |
| Geranlol | | | 1-5 | | | | | | | | | | | | | | | |

**Weight-% relative to total preparation [symbol]
indicates data missing or illegible when filed

1. A composition comprising adaptable vesicular aggregates characterized by:
at least one amphipat that forms adaptable vesicular aggregates with at least one amphipatic lipid bilayer in a polar fluid; and
at least one water-soluble additive with at least one C-atom and a tendency to accumulate near the aggregate surface to thereby improve at least one aggregate property, wherein said additive molecule provides:
at least a 20% faster reduction of the average aggregate size under an external stress, wherein said aggregate size-reduction is reflected in a corresponding turbidity decrease; or
at least a 20% greater ability of the aggregate to cross a constriction that is at least 50% narrower than the average aggregate diameter;
compared to an adaptable vesicular aggregate lacking said additive.

2. The composition of claim 1,
wherein the at least one water-soluble additive with at least one C-atom further comprises a net opposite charge to the charge of each of the at least second amphipat, and
wherein a second amphipat of the adaptable vesicular aggregate has an affinity for said aggregate and imparts a net charge on the aggregate surface.

3. A composition comprising adaptable bilayer vesicle aggregates characterized by:
wherein the at least one water-soluble additive with at least one C-atom further has an affinity for water, and
wherein said additive has a tendency to accumulate near the aggregate surface and thus to expand or partially destabilise the bilayer, and
wherein the at least one amphipat has about 12 or about 18 carbon atoms per fluid hydrophobic chain, each having an average area per chain (Ac) in a monolayer or bilayer of between about 0.35 nm² and about 0.55 nm².

4. A composition according to claim 1, wherein the adaptable vesicular aggregates further comprises:
at least one polar fluid; and
at least one water-soluble additive with at least one charge; and
at least one amphipat having at least one fluid, hydrophobic segment with nC carbon atoms, preferably about 5nC/24 to about 8.5nC/24 polarity units per hydrophobic segment, wherein said hydrophobic segment is attached to at least one hydrophilic headgroup in the at least one amphipat, and
wherein said at least one amphipat may be supplemented with one or more additional amphipats such that the total sum of all polarity units on all amphipats is about 8.5nC/24.

5. A composition according to claim 4, wherein the at least one amphipat has at least one net charge.

6. A composition according to claim 1, wherein the water-soluble and aggregate-improving additive has an octanol-water or aggregate-water distribution ratio of about −1±2.

7. A composition according to claim 1, wherein the water-soluble and aggregate-improving additive is used at a total concentration of between about 5 mM and about 600 mM.

8. A composition according to claim 1, wherein the total dry mass of aggregate forming components is between 0.05 wt.-% and 40 wt.-%

9. A composition according to claim 1, wherein the composition pH and/or the pH prevailing at a site of application of said composition provides at least 20% ionisation of the aggregates or of the additive itself, and preferably of both.

10. A composition according to claim 9, wherein the composition pH is in the range between about pH≦(pK_{a,mem}+0.5) and around pH≦(pK_{a,mem}−1.5) for aggregates with a positively charged surface, and between about pH≧(pK_{a,mem}−0.5) and around pH≧(pK_{a,mem}+1.5) for aggregates with a negatively charged surface.

11. A composition according to claim 1, wherein the average aggregate diameter is between 20 nm and 1 μm.

12. A composition according to claim 1, wherein the at least one amphipat is characterized by:
one or more hydrophobic segments acting together as hydrophobic anchors that are directly or indirectly attached to at least one hydrophilic headgroup, wherein the direct attachment can be by a fatty chain ether, ester or amide linkage and wherein the indirect attachment can be by a sorbitane-, glycerol-, or oligooxyethylene-mediated fatty-conjugate with an oligomer or a polymer of a polyalcohol or a sugar, or a sugar oligomer, or its chemically stable lactone linkage; or
a linear or branched fatty chain ester, ether, amide or sphingoside having a phospho-headgroup or sulpho-headgroup or
a fatty phosphonate or sulphonate, glycerophosphocholine, glycerophosphodimethylethanolamine, glycerophosphoserine, glycerophosphoglycerol, glycerophosphomethyl- or -ethyl-ester or glycerophosphate and glycerosulphate; or
a betaine or sulphobetaine, a fatty dimethyl-ammonio]-1-alkane sulphonic, -phosphonic, or -acetic acid; or
a fatty secondary, (di)fatty ternary or quaternary amine, a (di)fatty amino or imino acid or any of its suitable derivatives; or
a fatty amine-oxide.

13. A composition according to claim 1, wherein the hydrophobic segment on the amphipat has between about 8 and about 24 C-atoms.

14. A composition according to claim 1, wherein the composition is associated with at least one pharmaceutical agent.

15. A composition according to claim 14, wherein the at least one pharmaceutical agent is a drug useful for treating the digestive system, the cardiovascular system, the central nervous system, pain, musculo-skeletal disorders, the eye, the ear, nose, and oropharynx, the respiratory system, endocrine problems, the reproductive system or urinary system, the skin, infections and infestations, the immune system, allergic disorders, neoplastic disorders, or a drug for use in obstetrics and gynecology or for purposes of nutrition, contraception or diagnostic applications.

16. A composition according to claim 14, wherein the pharmaceutical agent is used for treating a skin condition.

17. A composition according to claim 14, wherein the pharmaceutical agent is an NSAID including ketoprofen, ketorolac, indomethacin, diclofenac, naprofen, ibuprofen, etoricoxib, celecoxib, rofecoxib, valdecoxib, meloxicam and piroxicam.

18. A composition according to claim 14, wherein the pharmaceutical agent is an antimycotic agent including allylamines, such as butenafine, naftifine, or terbinafine, candicin, imidazoles, such as bifonazole, butoconazole, clotrimazole, econazole, fenticonazole, isoconazole, ketoconazole, or miconazole plus omoconazole, oxiconazole, sertaconazole, sulconazole, tioconazole, triazoles, such as fluconazole, isavuconazole, or itraconazole plus posaconazole, ravuconazole, voriconazole, terconazole, amorolfine, griseofulvin, amphotericin B, filipin, hamycin, natamycin, nystatin, rimocidin, or abafungin.

19. A composition according to claim 1, wherein the at least one water soluble additive is a linear or a branched, at least partially ionised fatty acid or an organic, at least partially ionised, base that in either case carries from about 2 to about 7 C-atoms per charged group plus about 1.5-2.5 more C-atoms per every extra charged residue and every extra uncharged polar group, such as an oxo- or hydroxy-group, in the additive, and about 1-2 C-atoms less per each apolar group, e.g. a halide atom, in the additive.

20. (canceled)

21. (canceled)

22. A method of preparing the composition comprising adaptable vesicular aggregates according to claim 3, comprising:
selecting the at least one amphipat to have a molar ratio in the range of 0.1% to 50% with the at least one additive,
wherein said molar ratio is between 0.5% and 25% for additives that decrease the average area per hydrophobic chain of the amphipat, and
wherein said molar ratio is between 25% and 250% for additives that increase the average area per chain.

23. (canceled)
